(19) United States
(12) Patent Application Publication
(10) Pub. No.: US 2023/0181869 A1
(43) Pub. Date: Jun. 15, 2023

(54) MULTI-SENSORY EAR-WEARABLE DEVICES FOR STRESS RELATED CONDITION DETECTION AND THERAPY

(71) Applicant: Starkey Laboratories, Inc., Eden Prairie, MN (US)

(72) Inventors: Paul N. Reinhart, Minneapolis, MN (US); Justin R. Burwinkel, Eden Prairie, MN (US); Gerard N. Weisensel, Savage, MN (US)

(21) Appl. No.: 18/062,402

(22) Filed: Dec. 6, 2022

Related U.S. Application Data
(60) Provisional application No. 63/287,774, filed on Dec. 9, 2021.

(51) Int. Cl. A61M 21/02

(52) U.S. Cl. CPC A61M 21/02; A61M 2021/0027

(57) ABSTRACT

Embodiments herein relate to ear-wearable systems and devices that can detect and/or take actions to prevent or alleviate stress related conditions such as post-traumatic stress disorder (PTSD) and the like. In an embodiment, an ear-wearable stress therapy system is included having a control circuit, a first sensor package, a microphone, and an electroacoustic transducer, wherein the electroacoustic transducer is in electrical communication with the control circuit. The ear-wearable stress therapy system can be configured to initiate administration of desensitization and reprocessing (DR) therapy to a device wearer. Other embodiments are also included herein.

This application claims the benefit of U.S. Provisional Application No. 63/287,774, filed Dec. 9, 2021, the content of which is herein incorporated by reference in its entirety.

### FIELD

Embodiments herein relate to ear-wearable monitoring systems, devices and methods. Embodiments herein further relate to ear-wearable systems and devices that can detect and/or take actions to prevent or alleviate stress related conditions such as post-traumatic stress disorder (PTSD) and related conditions.

### BACKGROUND

Post-traumatic stress disorder (PTSD) is a mental disorder recognized in the DSM-5 (Diagnostic and Statistical Manual—American Psychiatric Association) that can develop after exposure to a traumatic event. It has been estimated that the lifetime prevalence of PTSD among adult Americans is approximately 6.8%. Traumatic events vary but can include sexual assault, warfare, traffic collisions, child abuse, threatened death, actual or threatened serious injury, and the like. Symptoms of PTSD can include upsetting memories, nightmares, flashbacks, mental or physical distress to trauma-related cues or triggers, attempts to avoid trauma-related cues or triggers, alterations in the way a person thinks and feels, and an increase in the fight-or-flight response. In many cases, some PTSD symptoms can be episodic and occur in response certain cues or triggers. For example, an individual may hear a car backfire and then experience PTSD symptoms and/or more intense PTSD symptoms.

PTSD can have many negative effects including overly negative thoughts and assumptions about oneself or the world, exaggerated blame of self or others for causing the trauma, negative affect, decreased interest in activities, feeling isolated, difficulty experiencing positive affect, irritability or aggression, risky or destructive behavior, hypervigilance, heightened startle reaction, difficulty concentrating, and difficulty sleeping.

### SUMMARY

Embodiments herein further relate to ear-wearable systems and devices that can detect and/or take actions to prevent or alleviate stress related conditions such as post-traumatic stress disorder (PTSD) and related conditions. In a first aspect, an ear-wearable stress therapy system can be included having a control circuit, a first sensor package, a microphone, and an electroacoustic transducer. The ear-wearable stress therapy system can be configured to initiate administration of desensitization and reprocessing (DR) therapy to a device wearer.

In a second aspect, in addition to one or more of the preceding or following aspects, or in the alternative to some aspects, the ear-wearable stress therapy system can be configured to administer and control delivery of binaural stimulation as part of the DR therapy.

In a third aspect, in addition to one or more of the preceding or following aspects, or in the alternative to some aspects, the binaural stimulation can include at least one of acoustic, visual, or haptic stimulation.

In a fourth aspect, in addition to one or more of the preceding or following aspects, or in the alternative to some aspects, the ear-wearable stress therapy system can be configured to monitor device wearer engagement with the binaural stimulation.

In a fifth aspect, in addition to one or more of the preceding or following aspects, or in the alternative to some aspects, the ear-wearable stress therapy system monitors engagement by directly sensing device wearer activity.

In a sixth aspect, in addition to one or more of the preceding or following aspects, or in the alternative to some aspects, the ear-wearable stress therapy system monitors engagement by indirectly monitoring sustained attention.

In a seventh aspect, in addition to one or more of the preceding or following aspects, or in the alternative to some aspects, the ear-wearable stress therapy system can be configured to automatically adjust properties of the binaural stimulation to maintain device wearer engagement.

In an eighth aspect, in addition to one or more of the preceding or following aspects, or in the alternative to some aspects, the ear-wearable stress therapy system can be configured to store data about the DR therapy and send the data to a care provider. In a ninth aspect, in addition to one or more of the preceding or following aspects, or in the alternative to some aspects, the ear-wearable stress therapy system can be configured to detect a physiologic stress response.

In a tenth aspect, in addition to one or more of the preceding or following aspects, or in the alternative to some aspects, the ear-wearable stress therapy system can be configured to detect a sympathetic nervous response.

In an eleventh aspect, in addition to one or more of the preceding or following aspects, or in the alternative to some aspects, the ear-wearable stress therapy system can be configured to detect post-traumatic stress disorder (PTSD) symptoms.

In a twelfth aspect, in addition to one or more of the preceding or following aspects, or in the alternative to some aspects, the ear-wearable stress therapy system can be configured to initiate administration of desensitization and reprocessing (DR) therapy to a device wearer subsequent to detection of PTSD symptoms.

In a thirteenth aspect, in addition to one or more of the preceding or following aspects, or in the alternative to some aspects, the ear-wearable stress therapy system can be configured to record microphone and/or sensor data over a look-back period when PTSD symptoms can be detected.

In a fourteenth aspect, in addition to one or more of the preceding or following aspects, or in the alternative to some aspects, the ear-wearable stress therapy system can be configured to use recorded microphone and/or sensor data to characterize at least one of a sound environment of the device wearer, a physical activity level of the device wearer, a conversation pattern of the device wearer, a level of irritability of the device wearer, an aggression level of the device wearer, and an emotional state of the device wearer.

In a fifteenth aspect, in addition to one or more of the preceding or following aspects, or in the alternative to some aspects, the ear-wearable stress therapy system can be configured to identify a triggering event selected from at least one a sound environment, a physical geolocation, a physical activity, and a conversation pattern.

In a sixteenth aspect, in addition to one or more of the preceding or following aspects, or in the alternative to some aspects, the ear-wearable stress therapy system can be configured to provide an avoidance recommendation to the device wearer based on the identified triggering event.

In a seventeenth aspect, in addition to one or more of the preceding or following aspects, or in the alternative to some aspects, the ear-wearable stress therapy system can be configured to compare the detected PTSD symptoms with previously detected PTSD symptoms of the device wearer to determine a trend.

In an eighteenth aspect, in addition to one or more of the preceding or following aspects, or in the alternative to some aspects, the ear-wearable stress therapy system can be configured to compare PTSD symptoms detected during waking hours for the device wearer with PTSD symptoms detected during sleeping hours for the device wearer.

In a nineteenth aspect, in addition to one or more of the preceding or following aspects, or in the alternative to some aspects, the detection of the PTSD symptoms can be based on at least one of a heart rate, a blood pressure, a physiologic temperature, the device wearer's voice, and a motion pattern.

In a twentieth aspect, in addition to one or more of the preceding or following aspects, or in the alternative to some aspects, the ear-wearable stress therapy system can be configured to detect medication administration events and determine a correlation between detected medication administration events and detected PTSD symptoms.

In a twenty-first aspect, in addition to one or more of the preceding or following aspects, or in the alternative to some aspects, the ear-wearable stress therapy system can be configured to titrate medication dosage based on an observed correlation between detected medication administration events and detected PTSD symptoms.

In a twenty-second aspect, in addition to one or more of the preceding or following aspects, or in the alternative to some aspects, the ear-wearable stress therapy system can be configured to provide audio stimulation when PTSD symptoms are detected.

In a twenty-third aspect, in addition to one or more of the preceding or following aspects, or in the alternative to some aspects, the audio stimulation can be sufficient to pull the device wearer out of a slow-wave sleep phase.

In a twenty-fourth aspect, in addition to one or more of the preceding or following aspects, or in the alternative to some aspects, the audio stimulation can be provided if the ear-wearable stress therapy system detects that the device wearer is in a slow-wave sleep phase.

In a twenty-fifth aspect, in addition to one or more of the preceding or following aspects, or in the alternative to some aspects, the ear-wearable stress therapy system can be configured to query the device wearer about their perceived stress level when PTSD symptoms are detected.

In a twenty-sixth aspect, in addition to one or more of the preceding or following aspects, or in the alternative to some aspects, the ear-wearable stress therapy system can be configured to issue an alert to a care provider when PTSD symptoms are detected.

In a twenty-seventh aspect, in addition to one or more of the preceding or following aspects, or in the alternative to some aspects, the ear-wearable stress therapy system can be configured to detect a PTSD symptom causing trigger.

In a twenty-eighth aspect, in addition to one or more of the preceding or following aspects, or in the alternative to some aspects, the ear-wearable stress therapy system can be configured to initiate administration of desensitization and reprocessing (DR) therapy to a device wearer when a PTSD symptom causing trigger have been detected.

In a twenty-ninth aspect, in addition to one or more of the preceding or following aspects, or in the alternative to some aspects, the ear-wearable stress therapy system can be configured to compare the PTSD symptom causing trigger with previously identified PTSD causing triggers.

In a thirtieth aspect, in addition to one or more of the preceding or following aspects, or in the alternative to some aspects, the ear-wearable stress therapy system can be configured to attenuate the PTSD symptom causing trigger by modulating an output from the electroacoustic transducer.

In a thirty-first aspect, in addition to one or more of the preceding or following aspects, or in the alternative to some aspects, the PTSD symptom causing trigger can be attenuated by reducing a volume of sound outputted by the electroacoustic transducer.

In a thirty-second aspect, in addition to one or more of the preceding or following aspects, or in the alternative to some aspects, the ear-wearable stress therapy system can be configured to detect a physiologic stress response to a stress-causing environmental stimulus.

In a thirty-third aspect, in addition to one or more of the preceding or following aspects, or in the alternative to some aspects, the ear-wearable stress therapy system can be configured to detect a physiologic stress response in the absence of a stress-causing environmental stimulus.

In a thirty-fourth aspect, in addition to one or more of the preceding or following aspects, or in the alternative to some aspects, the first sensor package can include a motion sensor, a temperature sensor, and a photoplethysmography sensor.

In a thirty-fifth aspect, in addition to one or more of the preceding or following aspects, or in the alternative to some aspects, the ear-wearable stress therapy system can be configured to detect a decrease in heart rate variability.

In a thirty-sixth aspect, in addition to one or more of the preceding or following aspects, or in the alternative to some aspects, the ear-wearable stress therapy system can be configured to detect a startle response.

In a thirty-seventh aspect, in addition to one or more of the preceding or following aspects, or in the alternative to some aspects, the ear-wearable stress therapy system can be configured to detect a startle response based on at least one of motion sensor signals, microphone signals, and photoplethysmography sensor signals.

In a thirty-eighth aspect, in addition to one or more of the preceding or following aspects, or in the alternative to some aspects, the ear-wearable stress therapy system can be configured to detect a trend in startle responses.

In a thirty-ninth aspect, in addition to one or more of the preceding or following aspects, or in the alternative to some aspects, the ear-wearable stress therapy system can be configured to generate a medication administration reminder.

In a fortieth aspect, in addition to one or more of the preceding or following aspects, or in the alternative to some aspects, the ear-wearable stress therapy system can be configured to provide a non-DR therapy to the device wearer in addition to the DR therapy.

In a forty-first aspect, in addition to one or more of the preceding or following aspects, or in the alternative to some aspects, the ear-wearable stress therapy system can be configured to provide at least one of a cognitive therapy and a talk therapy to the device wearer.

In a forty-second aspect, in addition to one or more of the preceding or following aspects, or in the alternative to some aspects, the ear-wearable stress therapy system can be configured to detect changes in a level of alcohol consumption of the device wearer.

In a forty-third aspect, in addition to one or more of the preceding or following aspects, or in the alternative to some aspects, the ear-wearable stress therapy system can be configured to detect changes in a level of device wearer self-talk.

In a forty-fourth aspect, in addition to one or more of the preceding or following aspects, or in the alternative to some aspects, the ear-wearable stress therapy system can be configured to detect a level of social engagement of the device wearer.

In a forty-fifth aspect, in addition to one or more of the preceding or following aspects, or in the alternative to some aspects, the ear-wearable stress therapy system can be configured to detect changes in the level of social engagement of the individual over time.

In a forty-sixth aspect, an ear-wearable stress therapy system can be included having a first ear-wearable device and a second ear-wearable device. The first ear-wearable device can include a first electroacoustic transducer, a first microphone, a first sensor package, and a first control circuit. The second ear-wearable device can include a second electroacoustic transducer, a second microphone, a second sensor package, and a second control circuit, wherein the ear-wearable stress therapy system can be configured to initiate administration of desensitization and reprocessing (DR) therapy to a device wearer.

In a forty-seventh aspect, a method of providing desensitization and reprocessing therapy to a device wearer can be included, the method including delivering the desensitization and reprocessing therapy to a device wearer with an ear-wearable device, and monitoring device wearer engagement with the desensitization and reprocessing therapy with the ear-wearable device.

In a forty-eighth aspect, in addition to one or more of the preceding or following aspects, or in the alternative to some aspects, delivering the desensitization and reprocessing therapy further includes administering and controlling delivery of binaural stimulation as part of the desensitization and reprocessing therapy.

In a forty-ninth aspect, in addition to one or more of the preceding or following aspects, or in the alternative to some aspects, the binaural stimulation can include at least one of acoustic, visual, or haptic stimulation.

In a fiftieth aspect, in addition to one or more of the preceding or following aspects, or in the alternative to some aspects, a method can further include adjusting properties of the binaural stimulation to maintain device wearer engagement.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of possible triggers or cues related to for conditions such as PTSD in accordance with various embodiments herein.

FIG. 2 is a schematic view of indicators of physiologic stress and anxiety in accordance with various embodiments herein.

FIG. 3 is a schematic view of an ear-wearable device in accordance with various embodiments herein.

FIG. 4 is a schematic view of an ear-wearable device within the ear in accordance with various embodiments herein.

FIG. 5 is a schematic view of an ear-wearable stress monitoring system in accordance with various embodiments herein.

FIG. 6 is a schematic view of an ear-wearable device in accordance with various embodiments herein.

FIG. 7 is a schematic view of an accessory device in accordance with various embodiments herein.

FIG. 8 is a schematic view is shown of device wearer interfacing with an external device in accordance with various embodiments herein.

FIG. 9 is a schematic frontal view of a subject wearing ear-worn devices in accordance with various embodiments herein.

FIG. 10 is a schematic view is shown of a pair of ear-worn devices disposed within the ear of a subject in accordance with various embodiments herein.

FIG. 11 is a schematic view of an ear-wearable device in accordance with various embodiments herein.

FIG. 12 is a schematic view of an ear-wearable device in accordance with various embodiments herein.

FIG. 13 is a schematic representation of possible triggers and detected PTSD symptoms along a timeline.

FIG. 14 is a schematic view of classification models in accordance with various embodiments herein.

FIG. 15 is a schematic view of classification models in accordance with various embodiments herein.

FIG. 16 is a block diagram view of components of an ear-wearable device in accordance with various embodiments herein.

FIG. 17 is a block diagram view of components of an accessory device in accordance with various embodiments herein.

FIG. 18 is a flow chart of operations associated with administration of a therapy herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein can include systems and devices that can detect stress-related conditions such as post-traumatic stress disorder (PTSD) symptoms and/or episodes as well as triggers for the same for individuals in their daily routines in real-life situations rather than in specialized laboratory settings. Embodiments herein further relate to ear-wearable systems and devices that can take actions to prevent or alleviate post-traumatic stress disorder (PTSD) and related conditions. For example, various ear-wearable systems and devices can provide therapies such as desensitization and reprocessing (DR) therapy.

Ear-wearable devices herein can function as multifunctional health devices that not only amplify sound to compensate for the wearer's hearing deficiencies, provide treatment for conditions such as tinnitus, and/or provide speech enhancement/augmented reality for individuals with typical hearing ability, but also measure multiple physiological parameters using embedded sensors. Systems and devices herein can apply algorithms to derive health insights related to stress from these data, including applying advanced machine learning techniques that are trained and validated with ground-truth data from representative groups of patients. Besides helping people hear better and tracking their physical health, the multi-sensory ear-worn devices herein can remotely and continuously detect PTSD triggers, symptoms and/or episodes and/or take actions to prevent or alleviate PTSD, symptoms thereof, and related conditions.

In various embodiments, the devices herein incorporate built-in sensors for measuring and analyzing multiple physiological and cognitive data to detect PTSD symptoms, including, but not limited to, core body temperature, cardiovascular parameters, including heart rate and heart rate variability, blood oxygen saturation (SpO2), blood pressure, electrodermal activities such as skin conductance determined from skin galvanic responses, sympathetic nervous stimulation, physical activities derived from accelerometers and gyroscopes, vocal biomarkers extracted from voice, respiratory events and rates, electroencephalographs (EEG), and electrocardiograms (ECG), amongst others. Data from these sensors, amongst other data utilized as described herein, can be processed by devices and systems herein to accurately gauge PTSD and related conditions and/or the symptoms thereof experienced by device wearers.

Machine learning models utilized herein are developed and trained with patient data, and deployed for on-device monitoring, classification, and communication, taking advantage of the fact that such ear-wearable devices will be continuously worn by the user, particularly in the case of users with hearing-impairment. In various embodiments, upon detection of the onset of symptoms related to PTSD and/or related conditions, and in some cases the assessment of the progression of the conditions, alleviations can be provided via appropriate channels including, for example, auditory stimulation, visual/optical stimulation, haptic stimulation, caloric stimulation, cochlear stimulation, neural stimulation, electromagnetic field/radiation stimulation, acoustic signals and instructions for remedial steps via the speakers embedded within the in-ear devices, desensitization and reprocessing (DR) therapy, and the like.

Further, recognizing that symptoms associated with PTSD vary from person to person, as well as the fact that reactions to possible triggers or cues vary highly amongst individuals, embodiments herein can include an architecture for personalization via on-device in-situ training and optimization phase(s).

In an embodiment, an ear-wearable therapy system is included having a control circuit; a sensor package, a microphone, and an electroacoustic transducer, wherein the ear-wearable stress therapy system is configured to initiate administration of desensitization and reprocessing (DR) therapy to a device wearer.

In various embodiments, an ear-wearable stress monitoring system herein can include a control circuit, a microphone, and a sensor package. The sensor package can include various sensors such as a motion sensor, as well as other sensors described herein. The ear-wearable system can be configured to evaluate data from at least one of the microphone and a sensor that is part of the sensor package and classify a level of symptoms being experienced by a device wearer using a machine learning classification model. In various embodiments, the system can also periodically update the machine learning classification model based on indicators of PTSD experienced by the device wearer and, thus, make the detection of PTSD and/or symptoms thereof personalized for the individual.

In various embodiments, an ear-wearable stress monitoring system is included having a control circuit, a microphone, a sensor package, and an electroacoustic transducer that can be configured for placement about or within an ear canal of the device wearer. The ear-wearable system can be configured to evaluate data from at least one of the microphone and the sensor package and classify a device wearer's condition with regard to PTSD and/or symptoms thereof using a machine learning classification model. The system can provide information to a clinician or care provider relating to the classified levels of PTSD and/or symptoms. In some embodiments, the system can further provide information to the device wearer relating to the classified levels of PTSD and/or symptoms thereof in a discrete manner so that the device wearer can receive information useful to them while keeping the information confidential with respect to others who may be near the device wearer.

In an embodiment, an ear-wearable stress monitoring system is included having a control circuit, a microphone, an electroacoustic transducer, and a sensor package. The ear-wearable system can be configured to detect PTSD and/or symptoms thereof exceeding a threshold value and evaluate data from at least one of the microphone and the sensor package over a lookback period to detect a likely trigger or cue for PTSD. In this way, the system/device can identify triggers or cues for PTSD that are specifically applicable for the device wearer.

In various embodiments, an ear-wearable system can be used to administer a therapy (such as desensitization and reprocessing (DR) therapy) by guiding the device wearer, controlling delivery of aspects of the therapy (such as bilateral and/or binaural stimulation), and monitoring device wearer engagement with the therapy (such as by evaluating sensor data).

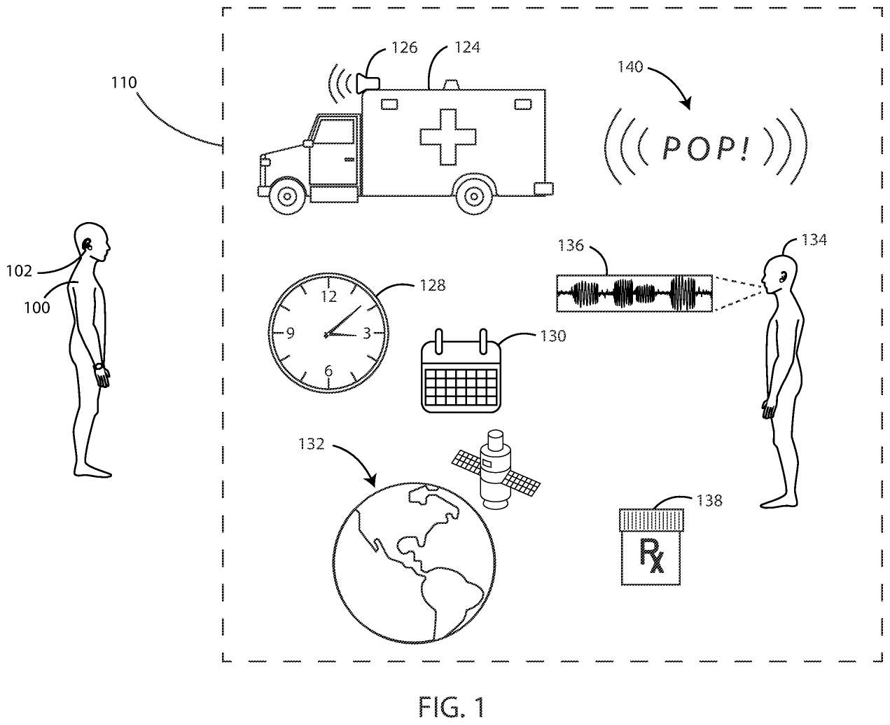

Referring now to FIG. 1, a schematic view is shown of aspects related to possible triggers or cues in accordance with various embodiments herein. In specific, FIG. 1 depicts a device wearer 100 with an ear-wearable device 102 that can be part of an ear-wearable system herein. Possible triggers or cues can include things such as objects making noise within the environment of the device wearer (ambient sounds) including, for example, vehicles 124 creating vehicle noise 126 which can be, without limitation, passenger vehicles, commercial vehicles, trucks, trains, heavy equipment, airplanes, and the like. Sources of ambient sounds can also include people, animals, other machinery, and the like. However, not all sounds are the same in terms of their triggering potential. For example, while particularly loud and sudden sounds (such as a gunshot or a loud pop 140) may be a likely trigger, sounds that are loud but more constant (such as noises associated with riding in a vehicle) may not be similarly triggering. However, such loud and constant noise may still result in adverse chronic stress. In various embodiments, the ear-wearable system can be configured to characterize the triggering potential of ambient sound by observing/detecting markers of PTSD and/or symptoms thereof occurring simultaneously with and/or subsequent to specific ambient sounds. Further, noise and/or subsonic/infrasonic vibrations, such as that resulting in or impacting wind turbine syndrome, vibroacoustic disease, or that experienced while living in a submarine can serve to lower the human threshold to other types of triggers or cues. In some embodiments, the ear wearable system can detect and can be configured to characterize the triggering potential of subsonic/infrasonic vibrations by observing/detecting markers of PTSD and/or symptoms thereof occurring simultaneously with and/or subsequent to subsonic/infrasonic vibrations.

In some embodiments, time 128 (such as time of day) can also be an item relevant for considering with respect to triggers or cues because susceptibility to triggers or cues can follow a diurnal pattern. For example, the steroid hormone cortisol is one of two main peripheral secretory products of the hypothalamic-pituitary-adrenal stress-neuroendocrine axis and typically follows a strong diurnal rhythm with levels are high on waking, surge in the 30-40 minutes after waking, drop rapidly in subsequent few hours after the awakening surge and then drop more slowly until reaching a nadir around bedtime. The levels of cortisol in a given individual may impact their reactions to possible triggers or cues. Thus, in various embodiments herein, the system/device can utilize information regarding the time of day to characterize an individual's PTSD symptoms as well as the individual's susceptibility to various possible cues or triggers. Further, cortisol levels (as a marker of stress, including chronic stress) themselves can be evaluated as a sign of PTSD symptoms.

The day of week or month, such as represented by calendar 130, can also be an item relevant for consideration/evaluation with respect to detecting possible triggers or cues for PTSD and/or symptoms thereof as it may relate to personal or work schedules, habits, activities, physiological cycles, and the like. For example, individuals may characteristically be more susceptible to triggers on Mondays if, for example, that represents the beginning of their working week. Also, individuals may be more susceptible to triggers over holidays such as when spent with extended family, or when time changes occur such as when daylight savings time begins or ends. Similarly, hormonal changes associated with menstrual cycles may impact susceptibility of some individuals to possible triggers. In various embodiments, the ear-wearable system can be configured to characterize the impact of the day of the week on potential triggers by observing/detecting patterns of potential triggers and simultaneous or subsequent markers of PTSD and/or symptoms thereof as a function of the day of the week (or other time measure) when the potential trigger and a response consistent with PTSD and/or symptoms thereof occurs.

The geolocation 132 of the individual can also be an item relevant for consideration/evaluation with respect to triggers for PTSD. In various embodiments, the ear-wearable stress and anxiety monitoring system can be configured to characterize the triggering potential of a specific geolocation 132 by observing/detecting markers of PTSD and/or symptoms thereof occurring simultaneously and/or subsequent to the presence of the device wearer at a specific geolocation 132. Geolocation 132 can be determined via a geolocation circuit of the system or device. Geolocation 132 can also be tracked in order to identify travel and/or time zone changes which may impact an individual's susceptibility to PTSD triggers. Related (in some instances) to geolocation is altitude. Changes in altitude, which can be associated with changes in geolocation, can impact an individual's susceptibility to PTSD triggers. Changes in altitude can be detected herein with a barometric pressure sensor.

Whether or not the device wearer has gotten sufficient sleep can greatly impact their physiological response to various possible triggers. Most individuals will generally experience worse reactions from a given possible trigger if they are lacking sufficient sleep than if they are well rested. As such, in various embodiments herein sleep can be evaluated as a piece of data along with other factors as described. For example, in various embodiments, the system and/or device can monitor and evaluate microphone and/or sensor data herein to detect patterns consistent with sleeping and record the same for evaluation along with other data herein when determining the presence of PTSD and/or symptoms thereof and/or evaluating whether or not specific possible triggers serve as triggers for the given device wearer. In some embodiments, the system and/or device can receive information regarding sleep from the device wearer as an input, from a third party as an input, from another device such as a sleep monitoring device, or from an electronic medical record system or the like.

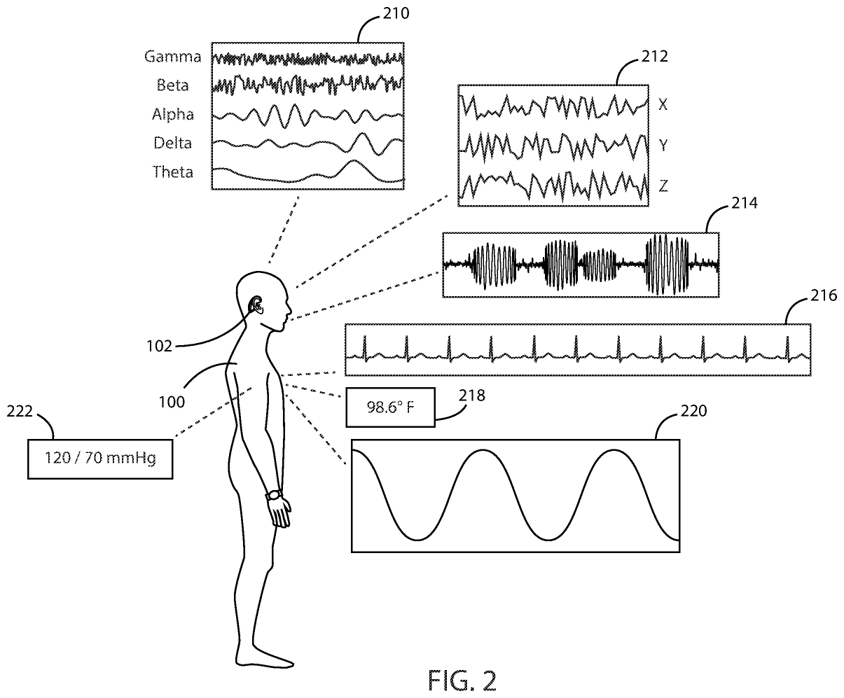

Possible triggers can also include various third parties 134 and/or their actions. FIG. 2 depicts a third party 134 along with third party communication 136. The third party 134 could be a family member, a care provider, a coworker, a manager, or the like. In various embodiments, the ear-wearable system can be configured to evaluate data from the microphone or other sensors to detect PTSD and/or symptoms thereof indicating the presence of an individual serving as a possible trigger. In various embodiments, the ear-wearable system can specifically be configured to evaluate data from the microphone to identify a voice of an individual serving as a trigger. It will be appreciated, however, that the system can also identify specific third parties through techniques other than analyzing voices. For example, electronic devices carried by third parties (devices personal to them such as smart phones or other personal electronics) can send advertising packets or other wireless data communications that can be received by the system or device(s) herein and can include data therein that can be used to identify a specific device and, by proxy, a specific third party. In various embodiments, the ear-wearable system can be configured to characterize the triggering potential of the presence of a specific third party 134 by observing/detecting markers of PTSD and/or symptoms thereof occurring simultaneously and/or subsequent to the presence of the specific third party 134.

Triggers can be highly variable amongst individuals. As such, in various embodiments, the ear-wearable system can be configured to learn what possible triggers generate a PTSD response for an individual by correlating possible detected triggers with subsequent PTSD and/or symptoms thereof of the device wearer 100. In various embodiments, the ear-wearable system can be configured to weight certain possible detected triggers in the classification model more heavily based on the correlation other statistical measures. In some embodiments, this weighting can be applied explicitly by the system/device. In some embodiments, this weighting can be applied through the generation of a machine learning model which includes such information as inputs.

The identity of the individual generating speech or other sounds can be tremendously important in determining whether specific speech or sounds are a trigger for the device wearer or a possible stressor that may lead to triggering the device wearer. For example, in various embodiments, the voice of the third party can be treated as a possible trigger. As such, in various embodiments, data from the microphone can be processed to distinguish a voice of the device wearer from a voice of a third party (exemplary aspects of own-voice detection are described in greater detail below).

Knowledge of meals can be an important consideration when detecting, monitoring, and/or predicting triggering events and/or symptoms of PTSD. In general, consuming a meal has the effect of reducing stress of an individual. However, this may not be consistently true for all individuals depending on their metabolism and other factors. Similarly, stress may rise along with the amount of time that has passed since a previous meal as the individual becomes hungry. As such, in various embodiments, the ear-wearable system can be configured to detect meals and correlate PTSD and/or symptoms thereof with detected meals and/or the amount of time that has passed since a previous meal.

Similar to meals, knowledge of medication events (e.g., when a device wearer takes or otherwise receives a medication 138) can be an important consideration when detecting triggers and/or PTSD symptoms. In some embodiments, the ear-wearable stress therapy system can be configured to detect medication administration events (through evaluation of sensor data and/or via response(s) to queries from the system to the device wearer) and determine a correlation between detected medication administration events and detected PTSD symptoms.

In some embodiments, the correlation between detected medication administration events and detected PTSD symptoms can be used to titrate medication dosages. For example, if the system detects that PTSD symptoms occur at a greater frequency during a time span after medication administration, then the system can provide a suggestion to the device wearer and/or a clinician to reduce the medication dosage. Conversely, if the system detects that PTSD symptoms occur at a reduced frequency during a time span after medication administration and thereafter the frequency increases, then the system can provide a suggestion to the device wearer and/or a clinician to increase the medication dosage.

In some scenarios, the drinking of fluids can be similar to food intake with respect to its impact on stress. In some cases, excessive food intake and/or insufficient hydration can be markers of increased stress and/or PTSD symptoms. In some cases, drinking of alcohol and/or changes in the same can be a PTSD symptom herein. In some embodiments herein, the device wearer can be queried regarding their consumption of alcohol and the answers to such queries can be used to determine levels of alcohol consumption along with changes in the same over time. In some embodiments, detection of drinking of a fluid (such as can be derived from microphone and/or motion sensor data herein) can be cross-referenced with location of intake (such as the geolocation of a tavern) to infer the consumption of alcohol. In some embodiments, symptoms of impairment from alcohol consumption can be identified by the device or system herein using sensor data so as to identify slurred speech (such as with microphone data) and/or changes in postural stability (such as with motion sensor data).

In various embodiments, the ear-wearable system can be configured to detect meals and/or fluid intake of the device wearer and cross-reference triggering events and/or PTSD symptoms against detected meals and/or fluid intake events. In some embodiments, meals or fluid intake events can be identified based on identifying or matching characteristic patterns in the data from a microphone and/or other sensors such as motions sensors herein. For example, a “positive” pattern for sensor data associated with a meal or a fluid intake event can be stored by the system and current data can be periodically matched against such a pattern. If a match exceeding a threshold value is found, then a meal or a fluid intake event can be deemed to have taken place. Further details regarding meal and fluid intake detection are provided in U.S. Pat. Appl. No. 63/058,936, titled “Ear-Worn Devices with Oropharyngeal Event Detection”, the contents of which are herein incorporated by reference in its entirety. Further details regarding medication event (such as taking or receiving a medication) detection are provided in U.S. Publ. Pat. Appl. No. 2020/0268315, titled “System and Method for Managing Pharmacological Therapeutics Including a Health Monitoring Device”, the contents of which are herein incorporated by reference in its entirety.

In various embodiments, the ear-wearable system can be configured to predict a future occurrence of a PTSD related event or symptoms of the device wearer 100. Such predictions can be based upon various factors including, but not limited to, those potential triggers described above and/or at least one of current inputs from the microphone and/or sensor package, information from a calendar of the device wearer, and a geographic location of the device wearer. Predictions can be particularly useful because predictions can allow suggestions or interventions to be issued by the system/device before the device wearer is currently in the midst of PTSD symptoms and distress. Further aspects of predictions are described in greater detail below.

In various embodiments, the system and/or device can detect many different possible markers of stress and/or signs that the individual is experiencing PTSD and/or PTSD symptoms. Referring now to FIG. 2, a schematic view is shown of some physiological indicators that can be detected in accordance with embodiments herein. In specific, FIG. 2 illustrates a device wearer 100 with an ear-wearable device 102. The ear-wearable device 102 can be used to directly sense and/or receive information regarding various sensed parameters. For example, the ear-wearable device 102 can include a sensor package (described in greater detail below) that can sense parameters including, but not limited to, electroencephalogram (EEG) data 210, accelerometer data 212 and/or gyroscope data from a motion sensor, the device wearer's voice 214, electrocardiogram (ECG) data 216 which can be used to determine heart rate and heart rate variability amongst other things, temperature data 218, respiratory data 220, and blood pressure data 222.

It will be appreciated, however, that the sensed parameters shown in FIG. 2 are only examples and that various other parameters and data are also contemplated herein. For example, sympathetic nervous stimulation may impact (typically reduce) urination. As such, urination events (which can be detected using microphone data, inputs from the device wearer or a third party, etc.) can also be used as data herein for the evaluation of stress and/or anxiety. As another example, electromyography (EMG) data can also be used herein as muscular tonus is often increased while an individual is experiencing distress. EMG data can be evaluated to sense, for example, auricular muscle contraction or tone and/or jaw muscle contraction or tone which can be used as data herein to detect distress. In some cases, eye movement and/or pupil dilation can be evaluated herein to detect distress. Aspects of detecting eye movement and pupil dilation as described in U.S. Publ. Pat. Appl. No. 2020/0143703, the content of which is herein incorporated by reference.

In various embodiments herein, the device wearer's voice 214 can be used to determine other factors bearing on whether they are experiencing PTSD symptoms. For example, the device wearer may experience differences in levels of self-talk when they are experiencing PTSD symptoms, such as greater amounts of self-talk. Self-talk can be identified through the device wearer's voice 214 by identifying words therein that are characteristic of self-talk. Self-talk can also be identified through the device wearer's voice 214 by identifying utterances that are made in the absence of any other voices (e.g., voices other than the device wearer's) being detected that would otherwise be consistent with a conversation. Aspects of own-voice detection are described elsewhere herein.

In various embodiments herein, the device wearer's voice 214 as well as the voices of others can be evaluated to determine levels of social engagement of the device wearer. In many cases, levels of social engagement of the device wearer would decrease as the device wearer is experiencing PTSD symptoms. In general, the more sequences of voices consistent with conversations (e.g., detection of the device wearer's voice followed by detection of another person's voice, or vice versa) the greater the social engagement level. It will be appreciated, however, that some individuals exhibit higher levels of social engagement as a baseline than others. As such, changes in levels of social engagement can be more important to detect than the absolute values for levels of social engagement. In various embodiments herein, the ear-wearable stress therapy system is configured to detect changes in the level of social engagement of the individual over time. Changes crossing a predetermined or dynamically determined threshold value can be treated as a PTSD symptom and/or marker of PTSD. For example, changes crossing a threshold value of 10, 20, 30, 40, 50, 75, 100, 150 percent or more compared with a baseline value for the individual can be treated as a PTSD symptom and/or marker of PTSD.

In various embodiments herein, startle responses (or startle reactions) can be used to identify PTSD and/or can be a PTSD symptom. A startle response frequently includes a jerky physical motion that can be detected by evaluating accelerometer data 212 and finding a signature of movement consistent with a startle response. In many cases, a startle response can be accompanied by an utterance or sound. Thus, microphone data can also be evaluated by the system in order to detect sound consistent with a startle response. In some embodiments, a startle response will also result in a characteristic signal from a photoplethysmography sensor. As such, in various embodiments herein, the ear-wearable system is configured to detect a startle response. In particular, the ear-wearable stress therapy system can be configured to detect a startle response based on at least one of motion sensor signals, microphone signals, and photoplethysmography sensor signals. In various embodiments, the ear-wearable stress therapy system can be configured to detect a trend in startle responses. In various embodiments, a trend of elevated or increasing numbers of startle responses and/or elevated or increasing intensity of the same can be deemed to be a symptom of PTSD.

For some individuals, they may experience PTSD symptoms more acutely and/or more intensely if they are asleep versus being awake. For others, the reverse may be true. In various embodiments herein, the ear-wearable stress therapy system can be configured to compare PTSD symptoms detected during waking hours for the device wearer with PTSD symptoms detected during sleeping hours for the device wearer. The system can determine whether an individual is sleeping or not based on sensor data herein. For example, the system can detect whether the individual is sleeping or not by evaluating EEG sensor data and detecting patterns characteristic of sleeping therein. In some embodiments, the system can detect whether the individual is sleeping or not by evaluating respiratory sensor data and detecting patterns characteristic of sleeping therein. Other sensor data can also be evaluated to detect whether or not the individual is sleeping.

In various embodiments, indicators of PTSD and/or PTSD symptoms experienced by the device wearer 100 can be derived from data produced by at least one of the microphone and the sensor package. In various embodiments, the sensor package can specifically include at least one including at least one of a heart rate sensor, a heart rate variability sensor, an electrocardiogram (ECG) sensor, a blood oxygen sensor, a blood pressure sensor, a skin conductance sensor, a photoplethysmography (PPG) sensor, a temperature sensor (to measure one or more of core body temperature, skin temperature, etc.), a motion sensor, an electroencephalograph (EEG) sensor, and a respiratory sensor. In various embodiments, the detection of the PTSD symptoms can be based on at least one of a heart rate, a blood pressure, a physiologic temperature, the device wearer's voice, and a motion pattern.

Acute parameters indicating PTSD and/or symptoms thereof can include, for example, one or more of (with respect to a baseline value) an increased heart rate, decreased heart rate variability, increased respiration rate, increased blood pressure, increased core temperature, changes in motion data such as that consistent with displacement behaviors and other nervous habits, and changes in the pitch (such as an increased pitch) and/or volume (such as an increased volume) of the device wearer's voice, amongst others. Baseline values for all of these parameters can be determined by the system/device over time as the ear-wearable device is being worn. Baseline values can be important to establish as these values are typically unique to individuals. For example, resting heart rates vary substantially across individuals, as well as the quantum of heart rate increase in response to stress. In various embodiments, the device can enter a baseline establishment mode where for a period of time spanning hours, days, weeks, or even months all of these types of data are tracked and then subjected to statistical operations in order to set baseline values.

Changes over baseline values deemed to have significance can be set as a default value, can be programmed in by the device wearer or a third party, or can be related to a statistical measure of baseline values such as in units of standard deviation. In various embodiments, changes over a baseline value of greater than or equal to 5, 15, 25, 35, 45, 55, 65, 75, 85, 95, 100, 150, or 200 percent, or an amount falling within a range between any of the foregoing, can be deemed to be a marker of an episode of PTSD and/or PTSD symptoms experienced by the device wearer. In some embodiments, the combination of measured parameters reflecting baseline or a non-stressed state can be compared with a current combination of measured parameters using machine learning approaches or other statistical approaches to determine whether the current state substantially matches the baseline state (e.g., the device wearer is not currently stressed or experiencing PTSD symptoms) or is different than the baseline state (e.g., the device wearer is currently experiencing stress or PTSD symptoms). In some embodiments, determinations can, in some cases, be binary (not stressed vs. stressed or no PTSD symptoms vs. PTSD symptoms). In other embodiments, determinations can be non-binary reflecting degrees of PTSD symptoms being experienced by the device wearer. In some embodiments degrees of PTSD symptoms can be relative to personal history of the device wearer and in other embodiments relative to normative data aggregated from a population or other individuals or device wearers. In various embodiments herein, data regarding measured parameters and other data can be used to classify a level of PTSD symptoms experienced by a device wearer using a machine learning classification model as described in greater detail below.

In various embodiments, the system can be configured to use recorded microphone and/or sensor data to characterize at least one of a sound environment of the device wearer, a physical activity level of the device wearer, a conversation pattern of the device wearer, a level of irritability of the device wearer, an aggression level of the device wearer, and an emotional state of the device wearer. Changes in a physical activity level of the device wearer, a conversation pattern of the device wearer, a level of irritability of the device wearer, an aggression level of the device wearer, and an emotional state of the device wearer can all be PTSD symptoms.

Depending on the individual, some of the acute physiological changes in response to stress or PTSD may be more prominent in some individuals versus others. As such, as described further below, systems and devices herein can adapt to an individual and thus more accurately detect the onset and/or changes in PTSD and/or symptoms thereof. Displacement behaviors, as one specific example, may be particularly subject to differences across individuals. As such, in various embodiments, the ear-wearable system can be configured to detect displacement behaviors and correlate PTSD symptom levels with detected displacement behaviors. For example, if for a given individual stress is frequently accompanied by a specific displacement behavior (such as one that is recognized by a particular pattern of a motion sensor or microphone such as shaking of their legs, arms, making certain noises, tapping, etc.) as revealed by a correlation between the two, then in future scenarios the detection of the displacement behavior can be effectively weighted more heavily, such that the system is more likely to categorize a level of stress as being significant or indicative of PTSD-related symptoms if the specific displacement behavior is also present. Correlations described herein can be derived using standard statistical technique that can show whether and how strongly pairs of variables and/or pairs of groups of variables are related.

In some embodiments, the ear-wearable system (described further below) can be configured to distinguish between acute and chronic symptoms of the device wearer 100. Markers of acute symptoms can include parameters such as increased heart rate, which is an almost-instantaneous sympathetic nervous response to stress. Markers of chronic symptoms can overlap with markers of acute stress but can also include markers that are largely distinct including trouble sleeping which can manifest, for example, as unusual movement detected by motion sensors during sleeping hours, increased background levels of displacement behaviors, fatigue which can manifest, for example, as decreased physical activity levels during waking hours, and the like. Embodiments herein can be configured to detect these markers of PTSD.

In various embodiments, the time scale of certain symptoms can be used to delineate between acute and chronic symptoms. For example, increased heart rate that rises transitorily then falls back to a normal level (unaccompanied by motion data consistent with physical exertion) is more likely to indicate acute symptoms. In contrast, a chronic elevation in heart rate is more likely to reflect chronic symptoms. Embodiments herein can be configured to calculate the time scale of changes in measured parameters that can be used to distinguish between acute and chronic symptoms. In some cases, those changes with a time scale crossing a threshold value can be deemed to be markers of chronic symptoms. The threshold value can be minutes, hours, days, weeks, or months. Therefore, the system/device can delineate between that which is an acute episode of symptoms which is experienced by the device wearer and changes in levels of chronic symptoms experienced by the device wearer.

In some examples, the specificity or sensitivity (or both) of detection operations herein can be improved by detection of an event or circumstance that is not necessarily related to PTSD symptoms, but nonetheless generates signals that could be confused with those reflecting stress and/or PTSD symptoms. For example, a device or system herein can determine an activity classification, such as running, sitting, or walking, and the activity classification can be considered as an input to an evaluation scheme herein. In an example, physiological changes (e.g., increased blood pressure, heart rate, or respiration rate) in a person who is sitting or walking slowly may correlate with PTSD symptoms, but if the person is actively exercising (e.g., running), such changes may be caused by physical exertion—not PTSD. Other data, such as geolocation data, may also be used in this manner. For example, the fact that a person is walking uphill (e.g., determined from geolocation) can be considered as a factor to evaluate the likelihood that a physiologic signals are related to PTSD symptoms as opposed to physical exertion.

In some embodiments, the system can record and/or analyze how the device wearer interfaces with the ear-wearable device during an episode of PTSD distress. For example, the device wearer may adjust operational parameters such as a gain value (controlling volume of sound provided by the device) up or down, may adjust ambient sound attenuation or cancellation settings, may make tinnitus masker stimulus volume adjustments, may turn tinnitus stimulus on or off, may change a tinnitus masker type, or any number of other changes. By recording such changes, the device can be configured to automatically make the same changes (or at least some of the same changes) the next time an episode of PTSD symptoms are detected so that the ear-wearable device automatically adjusts itself as desired by the device wearer during an episode of PTSD-related symptoms. In some embodiments, the device or system can query the user regarding the automatic adjustments to confirm their desirability. For example, in the case of automatically changing a tinnitus stimulus the device or system could pose a question to the device wearer such as “does this sound provide more relief?” Depending on the response from the device wearer, the change can either be kept or rolled back.

It can be important to understand the specific triggers of PTSD and/or PTSD symptoms for a particular individual. In various embodiments, the system and/or device can detect many different possible triggers or causes of triggers and, in some embodiments, empirically determine the impact of those triggers on the individual so that the relationship between possible triggers and PTSD and PTSD symptoms for a given individual can be elucidated. In this way, the device can customize detection as well as possible interventions to be most effective for a given individual. Conversely, where PTSD symptoms are detected, but no general stress response trigger (e.g., circumstances that would be expected to generate a stress response in a normal individual) is detected, this can be taken as an indication that the individual is suffering from PTSD or a related condition.

Ear-wearable devices herein, including hearing aids and hearables (e.g., wearable earphones), can include an enclosure, such as a housing or shell, within which internal components are disposed. Components of an ear-wearable device herein can include a control circuit, digital signal processor (DSP), memory (such as non-volatile memory), power management circuitry, a data communications bus, one or more communication devices (e.g., a radio, a near-field magnetic induction device), one or more antennas, one or more microphones, a receiver/speaker, a telecoil, and various sensors as described in greater detail below. More advanced ear-wearable devices can incorporate a long-range communication device, such as a BLUETOOTH® transceiver or other type of radio frequency (RF) transceiver.

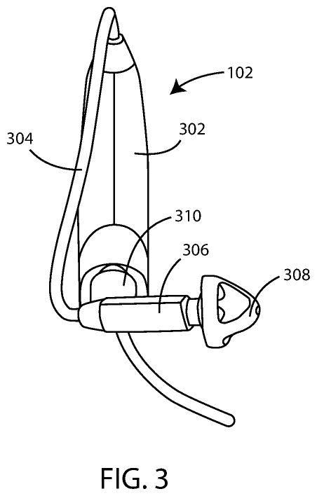

Referring now to FIG. 3, a schematic view of an ear-wearable device 102 is shown in accordance with various embodiments herein. The ear-wearable device 102 can include a device housing 302. The device housing 302 can define a battery compartment 310 into which a battery can be disposed to provide power to the device. The ear-wearable device 320 can also include a receiver 306 adjacent to an earbud 308. The receiver 306 an include a component that converts electrical impulses into sound, such as an electroacoustic transducer, speaker, or loudspeaker. Such components can be used to generate an audible stimulus in various embodiments herein. A cable 304 or connecting wire can include one or more electrical conductors and provide electrical communication between components inside of the device housing 302 and components inside of the receiver 306.

The ear-wearable device 102 shown in FIG. 3 is a receiver-in-canal type device and thus the receiver is designed to be placed within the ear canal. However, it will be appreciated that many different form factors for ear-wearable devices are contemplated herein. As such, ear-wearable devices herein can include, but are not limited to, behind-the-ear (BTE), in-the ear (ITE), in-the-canal (ITC), invisible-in-canal (ITC), receiver-in-canal (RIC), receiver in-the-ear (RITE), completely-in-the-canal (CIC) type hearing assistance devices, a personal sound amplifier, implantable hearing devices (such as a cochlear implant, a brainstem implant, or an auditory nerve implant), a bone-anchored or otherwise osseo-integrated hearing device, or the like.

While FIG. 3 shows a single ear-wearable device, it will be appreciated that in various examples, a pair of ear-wearable devices can be included and can work as a system, e.g., an individual may wear a first device on one ear, and a second device on the other ear. In some examples, the same type(s) of sensor(s) may be present in each device, allowing for comparison of left and right data for data verification (e.g., increase sensitivity and specificity through redundancy), or differentiation based on physiologic location (e.g., physiologic signal may be different in one location from the other location.)

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-wearable devices of the present disclosure can employ other radios, such as a 900 MHz radio. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a remote microphone device, a radio, a smartphone, a cell phone/entertainment device (CPED), a programming device, or other electronic device that serves as a source of digital audio data or files.

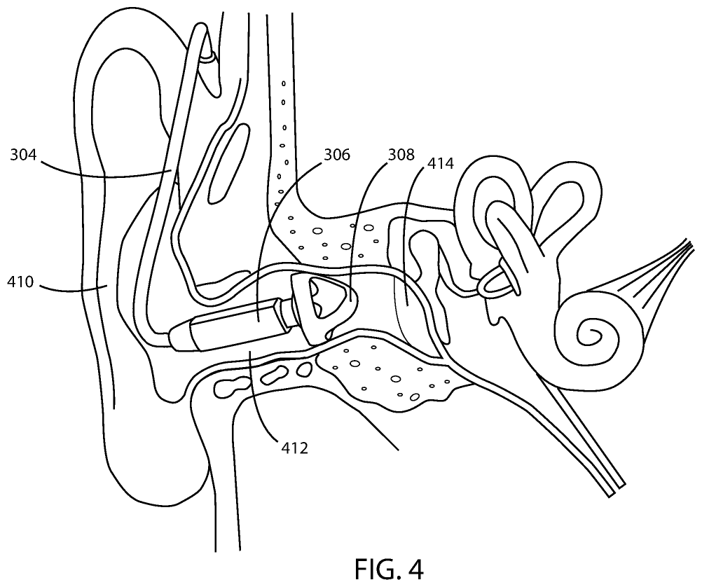

As mentioned above, the ear-wearable device 102 can be a receiver-in-canal (RIC) type device and thus the receiver is designed to be placed within the ear canal. Referring now to FIG. 4, a schematic view is shown of an ear-wearable device disposed within the ear of a subject in accordance with various embodiments herein. In this view, the receiver 306 and the earbud 308 are both within the ear canal 412, but do not directly contact the tympanic membrane 414. The hearing device housing is mostly obscured in this view behind the pinna 410, but it can be seen that the cable 304 passes over the top of the pinna 410 and down to the entrance to the ear canal 412.

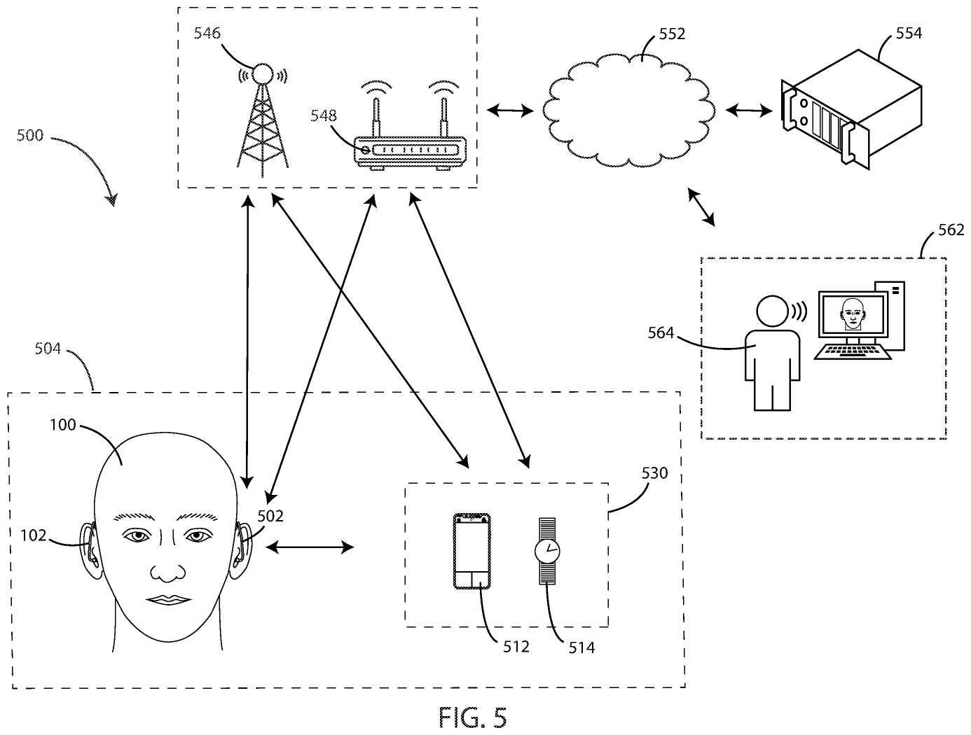

Referring now to FIG. 5, a schematic view of an ear-wearable stress monitoring system 500 is shown in accordance with various embodiments herein. FIG. 5 shows a device wearer 100 with an ear-wearable device 102 and a second ear-wearable device 502. The device wearer 100 is at a first location or patient location 504. The system can include and/or can interface with other devices 530 at the first location 504. The other devices 530 in this example can include an accessory device 512, which could be a smart phone or similar mobile communication/computing device in some embodiments. The other devices 530 in this example can also include a wearable device 514, which could be an external wearable device 514 such as a smart watch or the like.

FIG. 5 also shows communication equipment including a cell tower 546 and a network router 548. FIG. 5 also schematically depicts the cloud 552 or similar data communication network. FIG. 5 also depicts a cloud computing resource 554. The communication equipment can provide data communication capabilities between the ear-wearable devices 102, 502 and other components of the system and/or components such as the cloud 552 and cloud resources such as a cloud computing resource 554. In some embodiments, the cloud 552 and/or resources thereof can host an electronic medical records system. In some embodiments, the cloud 552 can provide a link to an electronic medical records system.

FIG. 5 also shows a remote location 562. The remote location 562 can be the site of a third party 564, which can be a clinician, care provider, loved one, or the like. The third party 564 can receive reports regarding the identified PTSD levels and/or PTSD symptoms of the device wearer. In some embodiments, the third party 564 can provide instructions for the device wearer regarding actions to take, such as actions to reduce or alleviate their PTSD symptoms. In some embodiments, the third party 564 can provide instructions for the device wearer regarding actions to take, such as actions to reduce or alleviate their PTSD symptoms. In some embodiments, the system can send information and/or reports to the third party 564 regarding the device wearer's condition and/or PTSD symptoms including trends and/or changes in the same. In some embodiments, the system can send information and/or reports to the third party 564 regarding therapy undertaken with the device wearer. In some embodiments, the system can send information and/or reports to the third party 564 regarding the device wearer's engagement with therapy, such as engagement with DR therapy. In some scenarios, information and/or reports can be sent to the third party 564 in real-time. In other scenarios, information and/or reports can be sent to the third party 564 periodically.

In various embodiments, the ear-wearable system 500 can be configured to send information regarding PTSD and/or PTSD symptoms to an electronic medical record system. In various embodiments, the ear-wearable system 500 can be configured to send information regarding classified stress levels to a third party 564. In some embodiments, the ear-wearable system 500 can be configured to receive information regarding stress, triggers, or PTSD-related events (such as testing information that may have been derived/performed in-clinic) as relevant to the individual through an electronic medical record system. Such received information can be used alongside data from microphones and other sensors herein and/or incorporated into machine learning classification models used herein.

In various embodiments, ear-wearable stress monitoring systems can be configured so that triggers of PTSD and/or PTSD symptoms are derived from inputs provided by a device wearer. Such inputs can be direct inputs (e.g., an input that is directly related to PTSD symptoms) or indirect inputs (e.g., an input that relates to or otherwise indicates a symptom of PTSD but indirectly). As an example of a direct input, the ear-wearable system can be configured so that a device wearer input in the form of a “tap” of the device can signal that the device wearer is experiencing distress. In some embodiments, the ear-wearable system can be configured to generate a query for the device wearer and the device wearer input can be in the form of a response to the query. As an example of an indirect input, because tinnitus may worsen when the device wearer is experiencing stress or anxiety the device wearer may make adjustments to tinnitus management settings on the system or device and this can be taken by the system as an indicator that they are experiencing PTSD or PTSD symptoms.

In some cases, the ear-wearable stress therapy system can take various steps to confirm the presence of PTSD symptoms and/or reduce false positives associated with the detection of PTSD symptoms. For example, in some embodiments the ear-wearable stress therapy system can be configured to query the device wearer about their perceived stress level when PTSD symptoms are detected. If the device wearer responds indicating that they do not perceive a significant stress level then this can be taken as an indication of a false positive by the system. Conversely, if the device wearer responds indicating that they perceive a significant stress level, then this can be taken as an indicator that PTSD symptoms are being experienced. In this manner, data from the sensors associated with such a response from the device wearer can be taken as a positive example of PTSD symptoms for use in supervised machine learning approaches as described in greater detail below.

In various embodiments, indicators of PTSD and/or PTSD symptoms experienced by the device wearer can also be received by the ear-wearable system from an external source.

Systems herein can take actions to prevent or alleviate post-traumatic stress disorder (PTSD) and related conditions. Preventative actions can include providing instructions to the device wearer (and/or a third party) regarding steps to take to prevent exposure to a trigger for PTSD symptoms. Triggers to avoid can be predetermined and/or programmed into the system, such as through input from a clinician or care provider. Triggers to avoid can also be derived by the system as it gathers data about the device wearer while being worn as described elsewhere herein. Triggers to avoid can specifically include any of those described with respect to FIG. 1 herein as well as others. For example, if a trigger for a given individual includes a specific geolocation (by virtue of the geolocation itself, someone in particular at the geolocation, or something occurring at the geolocation such as loud noises), then the system can provide a preventative instruction to the device wearer (audibly, visually, or haptically) to avoid going to that specific geolocation. In some cases, the preventative instruction can be provided to the device wearer if the system detects that their present location is within a threshold proximity of the geolocation serving as a trigger.

Preventative actions can also include providing steps to take to prevent an anticipated trigger from resulting PTSD symptoms or the exacerbation thereof. For example, the system can provide a preventative instruction to the device wearer telling them of steps to take to mitigate the possible effects of the trigger. As a specific example, the system can instruct the device wearer on proper breathing, provide positive messages, reminders of points that a clinician wants them to consider, etc.

Systems herein can also take actions to alleviate symptoms of post-traumatic stress disorder (PTSD) and related conditions. In some embodiments, the system can take actions based on what the device wearer is currently experiencing (e.g., reactive). By way of example, if the system determines that the individual is likely currently experiencing PTSD symptoms (such as described with respect to FIG. 2), then it can initiate actions to alleviate such symptoms. In some embodiments, such actions can include stimulation of various types such as caloric stimulation, nerve stimulation, auditory stimulation, electromagnetic field/radiation stimulation, haptic stimulation, optical stimulation, and the like. In some cases, such actions can include instructions to the device wearer to undertake a specific action such as a therapeutic action (such as breathing exercises), a prompt or reminder to take a medication, or the like. In some embodiments, the system can also take actions on a proactive basis (e.g., beyond what the device wearer is currently experiencing).

In some embodiments, the ear-wearable stress therapy system is configured to provide audio stimulation when PTSD symptoms are detected. The audio stimulation can take many different forms including spoken words, non-word sounds, music, and the like.

In some embodiments, audio stimulation provided can be sufficient to pull the device wearer out of a slow-wave sleep phase. This can be effective wherein the device wearer is one that tends to experience PTSD symptoms when they are sleeping. In some embodiments, the audio stimulation is provided if the ear-wearable stress therapy system detects that the device wearer is in a slow-wave sleep phase. Slow-wave sleep phases can be detected based on evaluation of EEG data herein. In such embodiments, when the system detects EEG signals consistent with a state of a slow-wave sleep phase and also detects symptoms of PTSD, such as those described herein, the system can provide audio stimulation at a volume sufficient to pull the device wearer out of the slow-wave sleep phase.

In some cases, the device can be configured to provide a therapy such as desensitization and reprocessing therapy (DR) and, specifically in some cases, eye movement desensitization and reprocessing therapy (EMDR). In some cases, the device can be configured to provide a therapy periodically (such as in specific sessions) over a period of time such as days, weeks, or months. DR therapy includes the use of alternating bilateral and/or binaural stimulation to re-stimulate the ability to process distressing events to allow the memory to become integrated into the patient's larger memory system which enables reduction of PTSD symptoms. In some embodiments, the system can administer DR in weekly, up to 90-minute individual sessions or as configured in the system by a clinician or care provider. In the course of DR therapy, the device wearer is instructed to focus on a target memory to be reprocessed and then the system leads the device wearer through and/or administers one or more bilateral and/or binaural stimulation sets.

Bilateral and/or binaural stimulation herein can include instructions provided to the device wearer regarding back and forth (or side to side) movement of the eyes or other body parts (e.g., “look to the left”, “now look to the right”), side to side tapping (“tap on your left side, now tap on your right side”). The back-and-forth movements can continue on to form a set of movements including 5, 10, 15, 20, 30, 45, 60, 120 movements or more, or a number of movements falling within a range between any of the foregoing.

Bilateral and/or binaural stimulation herein can also include bilateral stimulation directly provided to the device wearer by the system. For example, stimulation herein can include audio tones on the left side, then on the right side, etc., or tactile vibrations on the left side, then on the right side, etc. In various embodiments, bilateral and/or binaural stimulation herein can include a visual tracking task leading the eyes to ‘cross the midline’. A device wearer can be led in such tasks with audio and/or visual prompts as generated or initiated by the system. In some embodiments, an accessory or external device (as may be part of the system or to which the system may issue commands) can display a visual tracking task, such as a moving stimulus that the user will track with their eyes. In this instance, the device wearer can track the visual stimulus or object as it passes between their left and right visual fields in order to achieve bilateral stimulation. In various embodiments, bilateral and/or binaural stimulation herein can include stimulation (such as acoustic, electrical, or thermal stimulation) to alternating ears (e.g., left, right, left, right, etc.). The back and forth (left and right) stimulations of any type herein can continue on to form a set of stimulations including 5, 10, 15, 20, 30, 45, 60, 120 stimulations or more, or a number of stimulations falling within a range between any of the foregoing.

The system can be capable of providing the bilateral and/or binaural stimulation in accordance with various parameters. For example, in the context of auditory stimulation (acoustic or electrical), the stimulation can vary in one or more of frequency components, amplitude, modulation patterns, and/or frequency of alteration between devices (e.g., a left side device versus a right side device), amongst others. In some embodiments, the auditory stimulation may include one or more virtually spatialized sounds that perceptually oscillate from side to side, ‘crossing the midline’. For example, perceptual oscillation can be achieved with spatialized 3-D virtual sound as described further below. Haptic stimulation may include one or more of vibrotactile stimuli, electrical stimuli, or thermal stimuli. The haptic stimulation can vary in one or more of frequency, amplitude, and/or frequency of alteration between devices, among others. The visual stimulation can vary in direction of object movement, speed of movement, pattern of movement, and/or shape, color, size of visual stimulus being tracked, among others.

In various embodiments, the type and/or parameters of a given type of stimulation can be varied over time to ensure that the user is engaged with the stimulation (i.e., that the stimulation is taxing working memory). Various combinations of stimuli are envisioned and may be selectively used to regain or reinforce the attention of the user. Not to be bound by theory, an auditory metronome can be provided herein and can be useful for reinforcing attention to a visual oscillation stimulus, and/or a vibrotactile stimulation can be provided and can be useful for helping to regain the attention of a distracted user.

In some embodiments, the system can be configured to provide a non-DR therapy to the device wearer in addition to the DR therapy. For example, in some embodiments, the ear-wearable stress therapy system can be configured to provide at least one of a cognitive therapy and a talk therapy to the device wearer. In some embodiments, therapy can be provided through audio outputs delivered through an electroacoustic transducer (or speaker) of the ear-wearable device and/or as visual outputs as presented through an external device which could be, for example, a smartphone or a computing device such as a computer or tablet device. In some embodiments, therapy can be provided through haptic outputs delivered by an ear-wearable device of the system or another component of the system. In some embodiments, the outputs can include instructions for guided meditation.

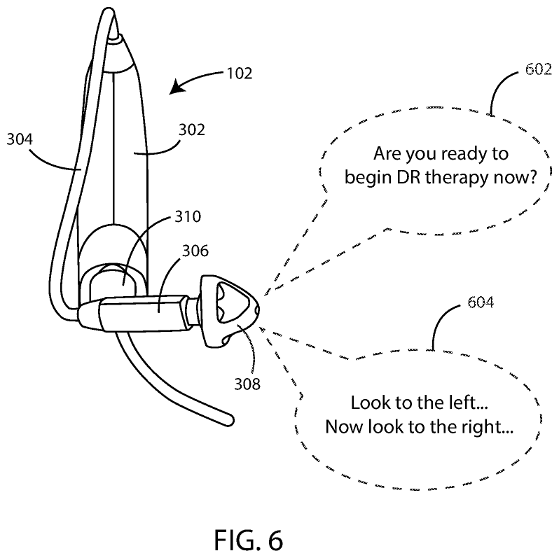

Referring now to FIG. 6, a schematic view of an ear-wearable device 102 is shown in accordance with various embodiments herein. The ear-wearable device 102 can be part of a system herein. The ear-wearable device 102 can include a housing 302, a cable 304, a receiver 306, an earbud 308, and a battery compartment 310. The ear-wearable system can be configured to generate and issue a wearer query 602. In some embodiments, the query 602 can be issued audibly by the ear-wearable device 102. However, by virtue of an electroacoustic transducer (or speaker) of the ear-wearable device 102 be positioned within or adjacent to the ear canal of the device wearer, the query 602 can be provided at a volume that can only be heard by the device wearer and thus discretely. The device wearer can respond to the query 602 in various ways. For example, in some embodiments, the device wearer can respond by way of a tap. In some embodiments, the device wearer can respond by way of a spoken answer that can be received by way of a microphone of the ear-wearable device. In some embodiments, the device wearer can respond by way of a specific gesture that can be identified by analyzing data from a motion sensor herein such as a head nod, head shake, or other head or body gesture.

In some embodiments, the query can specifically relate to a therapy session. For example, as shown in FIG. 6, the query 602 could relate to whether the device wearer is ready to begin a therapy session. If the reply to the query is affirmative, then in various embodiments the ear-wearable device 102 can provide prompts 604 for bilateral stimulation associated with DR therapy.

In some embodiments where the device wearer has an ear-wearable device in both ears, prompts 604 can be provided at an equal volume or intensity on both sides (for example, the instruction “look to the left” can be provided at an equal volume through a first ear-wearable device 102 and a second ear-wearable device 502). However, while not intending to be bound by theory, it is believed that bilateral stimulation can be enhanced by providing prompts such that the volume or intensity is not equal on both sides (e.g., the prompts for bilateral stimulation are themselves provided in a manner that can provide a bilateral stimulation benefit). As such, in some embodiments, the instruction “look to the left” can be provided at a greater intensity or volume (or be provided exclusively) through the left ear-wearable device whereas the instruction “look to the right” can be provided at a greater intensity or volume (or be provided exclusively) through the right ear-wearable device

In some embodiments, the sound delivered from the ear-wearable devices can be manipulated so as to be perceived as coming from the left side or from the right side (for purposes of queries or prompts and/or for purposes of audio stimulation). For example, systems herein can be configured to synthesize three-dimensional (3-D) audio that generates audio output comprising spatialized 3-D virtual sound emanating from virtual spatial locations that can be on one side or the other of the device wearer. The sound generated at the virtual spatial locations can be any broadband sound, such as complex tones, noise bursts, human speech, music, etc. or a combination of these and other types of sound. Further aspects of virtual audio interfaces are described in commonly owned U.S. patent application Ser. No. 15/589,298, titled “Hearing Assistance Device Incorporating Virtual Audio Interface for Therapy Guidance”, the content of which is herein incorporated by reference in its entirety.

In some embodiments herein, queries, prompts, and/or stimulation for the device wearer can be generated and/or issued to the device wearer using a different device. For example, in some embodiments, an accessory device can be used to present a query to the device wearer.

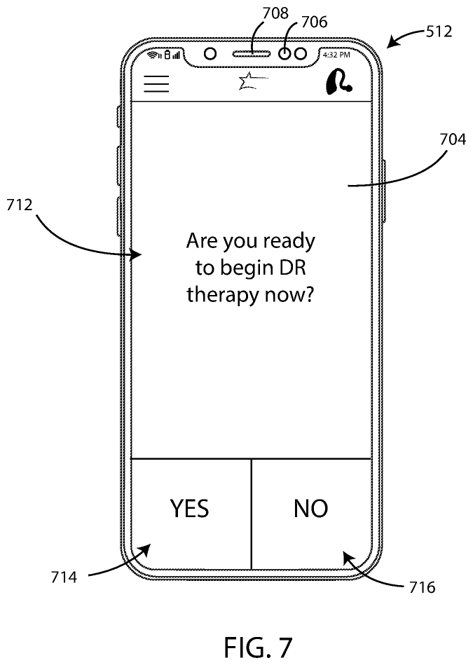

Referring now to FIG. 7, a schematic view of an accessory device 512 is shown in accordance with various embodiments herein. The accessory device 512 includes a display screen 704. The accessory device 512 also includes a camera 706 and a speaker 708. The accessory device 512 can generate and/or present an accessory query 712 or alternatively a prompt or instruction. In order to receive input from the device wearer, the accessory device 512 can also include, for example, a first user input object 714 and a second user input object 716.

In various embodiments herein, the ear-wearable system can be configured to provide various pieces of information to the device wearer relating to PTSD and/or detected symptoms thereof. In many embodiments herein, the ear-wearable system can be configured to provide information and/or instructions to the device wearer in a discrete manner. For example, in various embodiments, the ear-wearable system can provide information and/or instructions related to PTSD or symptoms thereof through an electroacoustic transducer at volume that only the device wearer can hear. The information provided to the device wearer relating to PTSD and/or symptoms thereof can take many forms. In some embodiments, the information can comprise verbal information. In some embodiments, the information provided to the device wearer can be provided via non-verbal sound(s). By way of example, in various embodiments, non-verbal sounds provided by the ear-wearable device can include music. In various embodiments, the information relating to PTSD or symptoms thereof includes a sound preselected by the device wearer.

In various embodiments herein, the system can be configured to monitor device wearer engagement with a therapy, such as device wearer engagement with binaural stimulation. Engagement can be measured in various ways. In some embodiments, the system can monitor engagement by directly sensing device wearer activity, such as sensing movements or actions associated with binaural stimulation and/or whether or not the device wearer is reacting to instructions within a threshold amount of time. In some embodiments, the system can monitor engagement by indirectly monitoring sustained attention, such as whether or not the device wearer is exhibiting behaviors consistent with distraction.

In accordance with various embodiments herein, the ear-worn device and/or the system can track movement of the subject's eyes using one or more of a camera, an EOG (electrooculogram) sensor, an EMG sensor, a VOG sensor, a motion sensor or another device. Such sensors can be a part of the ear-wearable device, device system, and/or part of another device in communication with the system. Movement of the subject's eyes can be used to measure device wearer engagement with binaural stimulation. As an example, if the device wearer is being prompted to move their eyes from side to side then the system can measure engagement with such prompts by evaluating whether the device wearer's eyes are in fact moving from side to side. If the wearer's eyes are not moving, moving less than a threshold value, and/or moving less than they were previously then this can be taken as a sign of decreased or decreasing engagement.

In some embodiments, the system can be configured to automatically adjust properties of the binaural stimulation to maintain and/or maximize device wearer engagement. For example, in some embodiments, the system can increase or decrease an intensity, volume, or pace of binaural stimulation if the detects that engagement with the therapy is dropping. In some embodiments, the system can automatically adjust one or more properties of the binaural stimulation if it determines that engagement is changing (or decreasing) and/or crosses a threshold value. For example, properties of the binaural stimulation that can be varied include the volume, intensity, and/or pace thereof.

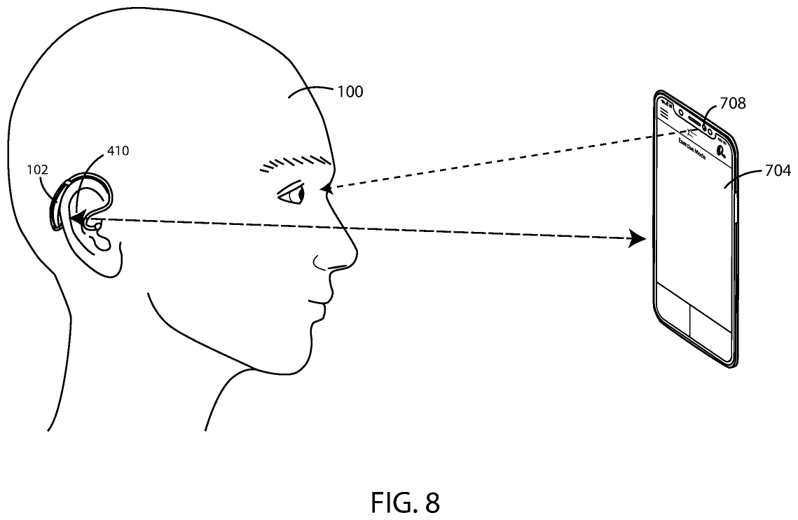

Referring now to FIG. 8, a schematic view is shown of device wearer 100 interfacing with an external device 804 in accordance with various embodiments herein. The external device 804 can include a display screen 806 and a camera 808. In some embodiments, the display screen 806 can be a touch screen. The display screen 806 can display various pieces of information to the device wearer 100 including, but not limited to, instructions for procedures to follow, visual feedback, information regarding the progress of the device wearer 100 through a particular set of procedures, or the like.

The camera 808 can be positioned to face toward the device wearer 100 (in some embodiments, the camera could also be facing the display, with the subject between the camera and the display screen using the display itself as a spatial reference). The camera 808 can be used to capture an image or images of the device wearer's 100 eyes. In some embodiments, the camera 808 can be used to capture image(s) including the positioning of device wearer's 100 face, pupil, iris, and/or sclera. Such information can be used to measure device wearer engagement with binaural stimulation.

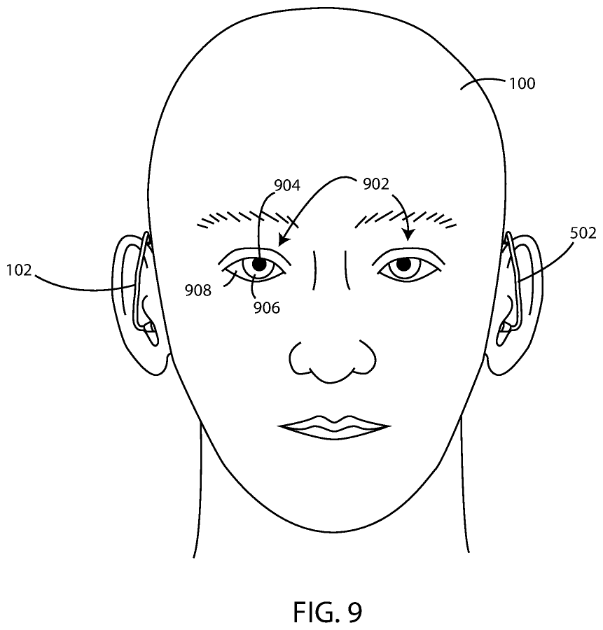

Referring now to FIG. 9, a schematic frontal view is shown of a device wearer 100 wearing ear-wearable devices 102, 502 in accordance with various embodiments herein. The device wearer's 100 eyes 902 include pupils 904, iris 906, and sclera 908 (or white portion). Identifying the position of these and other eye components and facial components can be used to determine the direction of gaze and/or direction the face is pointing as described above. In some embodiments, the size of the pupils 904 can be monitored using camera data to detect any changes that occur during an activity of the user. In some embodiments, eye movement can be tracked to detect engagement with therapy.

In some embodiments, engagement with therapy (such as engagement with a visual tracking task) can be detecting by monitoring ocular muscle activity. For example, in some embodiments, EOG/EMG sensor recordings can have values a right gaze, a center gaze, and/or a left gaze. In some embodiments, engagement with therapy can be detected by detecting movement with a motion sensor herein as the device wearer exhibits slight horizontal head movements corresponding to a visual tracking task. In some embodiments, one or more approaches for detecting device wearer engagement can be implemented independently or in any combination.

In some embodiments, information from other sensors (such as an EOG sensor) can be used solely or in combination with data from the camera to more accurately determine device wearer engagement with binaural stimulation and/or calculate the direction of the subject's face, gaze, eye movement or another aspect described herein. Aspects of EOG sensors are described in U.S. Pat. No. 9,167,356, the content of which is herein incorporated by reference in its entirety.

In various embodiments, when signals from sensors herein regarding device wearer engagement with therapy do not correlate with the therapy task (such as the current visual tracking task), then a determination can be made that the user is not engaged with the visual bilateral and/or binaural stimulation. In that scenario, the system can alter one or multiple properties of the stimulation (e.g., direction of object movement, speed of movement, or pattern of movement, among others) to reengage the user. For example, if the user's horizontal eye movements are lagged relative to the visual tracking task, then the speed of movement can be slowed so that the user can better track the visual stimulus.

In some embodiments, a method can include monitoring engagement by indirectly monitoring sustained attention and/or engagement with a therapy. For example, operatively connected devices and/or sensors (such as a camera, microphone, EEG, PPG, ECG, and GSR sensors) can be used to indirectly monitor sustained attention to a therapy or therapy step, such as bilateral stimulation. These devices and/or sensors can be used to monitor psychophysiological correlates of sustained attention, such as decreased heart rate, reduced respiratory variability, decreased skin conductance, and changes to various spontaneous neural oscillations. Data from one or multiple the devices and/or sensors herein can be used to detect these psychophysiological correlates. For example, respiratory variability can be detected acoustically via the microphone or by monitoring respiratory sinus arrhythmia via ECG and/or PPG sensors.

When sensor data indicates that a device wearer's psychophysiological profile is no longer indicative of them maintaining sustained attention, then the type and/or parameters of one or more stimulation pattern and stimuli combinations can be automatically varied by the system, such as until sustained attention is achieved and is confirmed using the previously described sensing techniques.

In some embodiments, sensor data can also be used to terminate the therapy process as a fail-safe. As an example, recall of the traumatic, target memory during DR therapy may trigger intense stress, and the process can be aborted to re-ground the user. For example, if sensor data indicates the user is an overly-stressed state (e.g., heart rate >120 bpm, vocal qualities change more than a threshold amount, etc.), then the system can terminate or modify the DR treatment in order to bring the user to a more relaxed state and/or provide an alert or notification to a third-party (e.g., caregiver, medical professional, etc.).

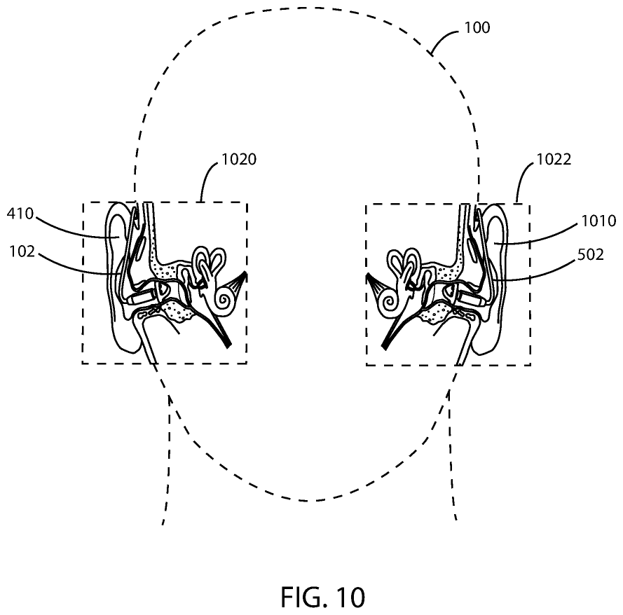

Referring now to FIG. 10, a schematic view is shown of a pair of ear-wearable devices 102, 502 disposed within the ear of a device wearer 100 in accordance with various embodiments herein. FIG. 10 further shows a portion of the right side 1020 of the vestibular system and a portion of the left side 1022 of the vestibular system. The first ear-wearable device 102 is worn about the right side pinna 410 (right side of the device wearer) and the second ear-wearable device 502 is worn about the left side pinna 1010.

Some embodiments herein may only use a single ear-worn device. The single ear-worn device can be used to provide stimulation (excitatory or inhibitory) such as caloric stimulation, nerve stimulation, auditory stimulation, electromagnetic field/radiation stimulation, haptic stimulation, optical stimulation, and the like. However, in various embodiments, two ear-worn devices are used and can provide bilateral stimulation to the left side 1022 of the vestibular system and the right side 1020 of the vestibular system. In some embodiments, the two ear-wearable devices 102, 502 can be used to provide differential bilateral stimulation (excitatory or inhibitory) such as differential caloric stimulation, differential nerve stimulation, differential auditory stimulation, differential electromagnetic field stimulation, differential optical stimulation, and the like. Exemplary aspects of stimulation are described in greater detail below.

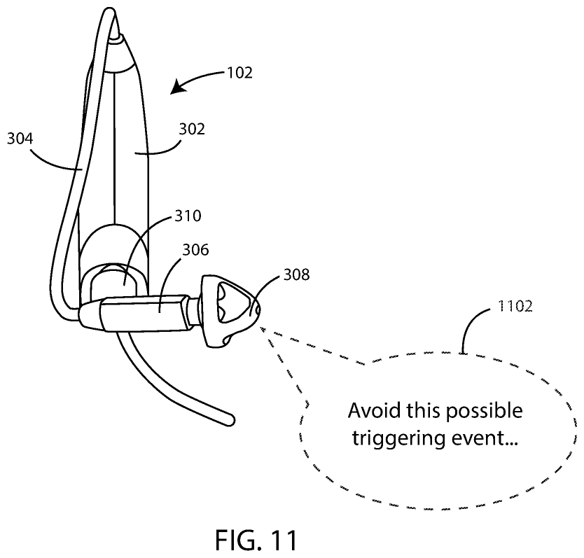

As described above, the system can take preventative actions such as providing instructions to the device wearer (and/or a third party) regarding steps to take to prevent exposure to a trigger for PTSD symptoms. Triggers to avoid can be predetermined and/or programmed into the system, such as through input from a clinician or care provider. Triggers to avoid can also be determined by the systems as specific for a given device wearers described elsewhere herein. Referring now to FIG. 11, a schematic view of an ear-wearable device 102 is shown in accordance with various embodiments herein. The ear-wearable device 102 can be part of an ear-wearable stress monitoring system. The ear-wearable device 102 includes a housing 302, a cable 304, a receiver 306, an earbud 308, and a battery compartment 310. The ear-wearable system can be configured to provide information 1102 to the device wearer, such as in a discrete manner. In various embodiments, the information relating to PTSD, symptoms thereof, instructions, or queries can be provided through an electroacoustic transducer that can be part of the receiver 306. In some embodiments, the information 1102 can specifically include a suggestion to avoid a particular possible triggering event, such as avoiding a particular geolocation.

In various embodiments, the information 1102 can specifically include breathing instructions. Aspects of breathing exercises and instructions can be found in U.S. Publ. Appl. No. 2020/0008708, the content of which is herein incorporated by reference. In various embodiments, the information 1102 can include a suggestion to meditate. In various embodiments, the information 1102 can include a suggestion to consume a food item. In various embodiments, the information 1102 can include a suggestion to take a medication.

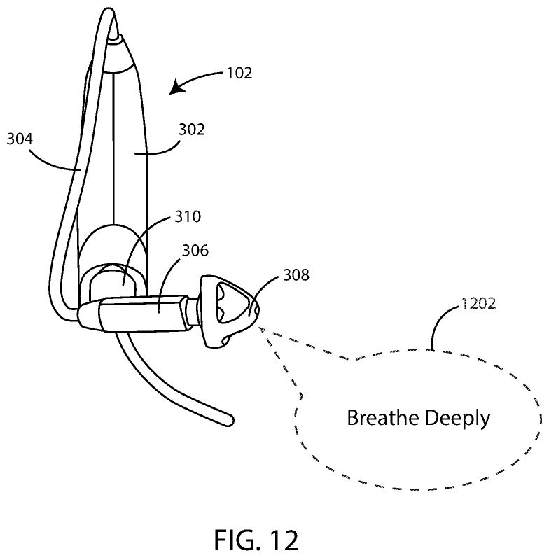

Referring now to FIG. 12, a schematic view of an ear-wearable device 102 is shown in accordance with various embodiments herein. The ear-wearable device 102 can be part of a system. The ear-wearable device 102 includes a housing 302, cable 304, receiver 306, earbud 308, and a battery compartment 310. The ear-wearable system can provide an instruction or suggestion 1202.

In various embodiments, the ear-wearable system can be configured to correlate possible detected stressors with subsequent classified stress levels of the device wearer to elucidate cause and effect relationships. Then this data can be used in various ways. For example, in some embodiments, the ear-wearable system can be configured to weight certain possible detected stressors in the machine learning classification model more heavily based on an identified correlation between a particular stressor and resulting stress that holds true for the particular individual wearing the device. In some embodiments, such correlations can be used in order to predict future stress.

In various embodiments, the ear-wearable system can be configured to detect an occurrence of symptoms of PTSD of the device wearer exceeding a threshold value. In various embodiments, the ear-wearable system can be configured to evaluate data from at least one of a microphone and a sensor package over a lookback period to detect a trigger of the PTSD symptoms exceeding a threshold value.

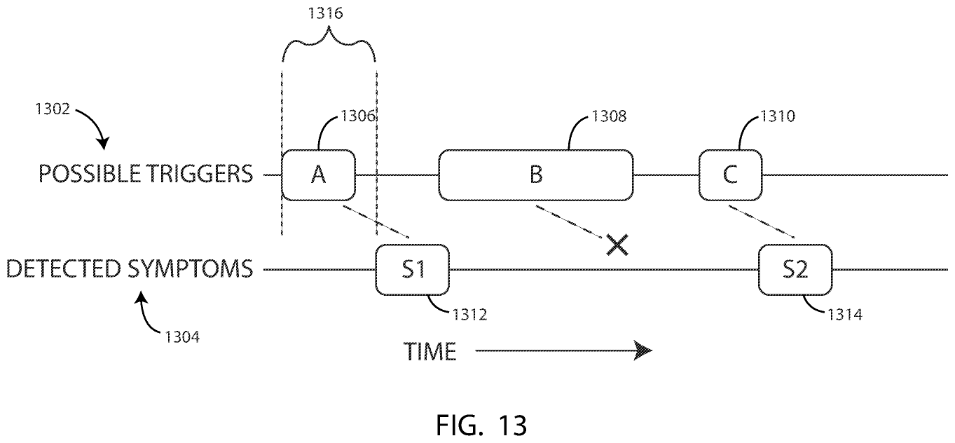

Referring now to FIG. 13, a schematic representation is shown of possible triggers 1302 and detected symptoms 1304 along a timeline. In this example, the possible triggers 1302 including trigger “A” 1306, “B” 1308, and “C” 1310. The detected symptoms 1304 includes episodes “S1” 1312 and “S2” 1314 that exceed a threshold value.

When a symptom episode is detected, the system can evaluate data from at least one of a microphone and a sensor package over a lookback period 1316. In this example, trigger “A” 1306 falls within the lookback period and this can be taken as an indication that trigger “A” 1306 may be a possible trigger that actually results in PTSD symptoms for the device wearer. To facilitate such operations, the device can be configured to store data for a rolling window of time reflecting the desired lookback period 1316.

In some embodiments, the lookback period 1316 can be greater than or equal to 5 seconds, 10 seconds, 30 seconds, 1 minute, 10 minutes, 20 minutes, 30 minutes, 40 minutes, 50 minutes, or 60 minutes, or can be an amount falling within a range between any of the foregoing.

In the example of FIG. 13, possible trigger “B” 1308 does not fall within a lookback period of any detected PTSD symptom episode, thus possible trigger “B” 1308 is not an actual trigger for the individual wearing the device. Further, symptom episode S2 1314 appears to be linked with possible trigger “C” 1310 as it falls within the lookback period for symptom episode S2 1314. In this manner, the device can determine which possible triggers act as actual triggers for the specific individual wearing the device and which do not, thereby customizing the monitoring, detection, and prediction capabilities of the system/device for the particular individual. For example, referring again to FIG. 13, if possible trigger “A” 1306 is observed (which could be any of the potential triggers described herein as well as others) or seen to be imminent (such as a meeting, travel to a specific location, meeting with a specific person, etc.) then the system can predict the onset of an episode of PTSD symptoms. As such, in some embodiments, the system can issue recommendations in advance to help the device wearer to either avoid the trigger or be in the best position to handle the expected episode of PTSD symptoms.

In some embodiments, information regarding relationships between triggers and PTSD symptom episodes can be reported to the device wearer and/or to a third party. In some embodiments, relationships between triggers and PTSD symptom episodes and other aspects (such as a worsening of tinnitus) can be analyzed and/or reported to the device wearer and/or a third party. For example, in some individuals an increased level of PTSD symptoms can cause a worsening of tinnitus, or vice-versa (a louder perceived tinnitus may increase anxiety/stress). The result of such an analysis can then be delivered to the user, caregivers, or hearing-care providers (e.g., “We noticed that your tinnitus gets worse when you are in situation X. You should consider trying to avoid situation X.” As another example, “We noticed that you get anxious when your tinnitus becomes louder. Did you know that your hearing device can play sounds that temporarily mask your tinnitus?” if the device wearer is not already using such operational features.

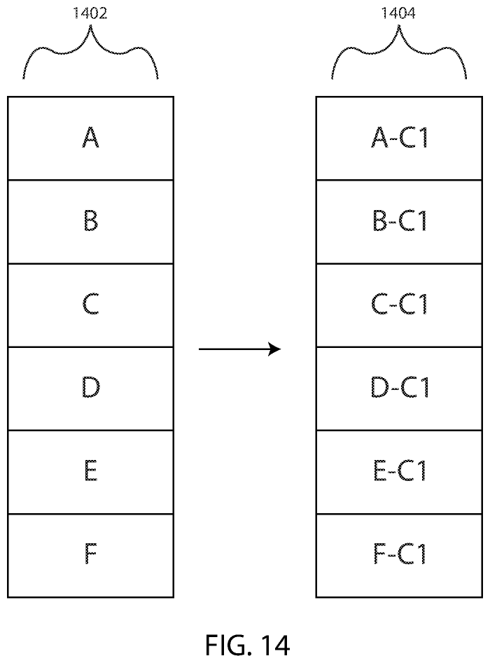

Referring now to FIG. 14, a schematic view is shown of classification models in accordance with various embodiments herein. An ear-wearable system can include and/or utilize a first or default machine learning classification model 1402. In this example, the ear-wearable system also includes a customized classification model 1404, wherein the customized classification model 1404 is specific for the device wearer and is created over time as the device wearer utilizes the system.

The system can utilize data from any of the sensors described herein and/or any of the sources of data described herein (e.g., indicators of PTSD and/or symptoms thereof) in a machine learning approach to categorize a current level of PTSD symptoms being experienced by the device wearer. For example, the ear-wearable system can be configured to evaluate data from at least one of the microphone and the sensor package and classify a PTSD symptom level of a device wearer 100 using a machine learning classification model 1402 and periodically update the machine learning classification model to generate a second or customized machine learning classification model 1402 based on indicators of PTSD symptoms experienced by the device wearer.

In some embodiments, the initial or default machine learning classification model can be generated using sets of data gathered from individuals numbering in the hundreds, or thousands, or more. The initial or default machine learning classification model can be generated using supervised or unsupervised machine learning approaches.

In various embodiments, the ear-wearable system (described further below) can be configured to weight certain possible detected triggers in the machine learning classification model 1402 more heavily based on the correlation.

In some embodiments, the system can more accurately sense PTSD symptoms if a model is used that is specific for individuals sharing some characteristics with the individual wearing the ear-wearable device. For example, a model can be used wherein the model is specific for individuals of a certain gender falling within a specific age range. Many other factors can be used including, for example, health status, weight, medical history, and the like.

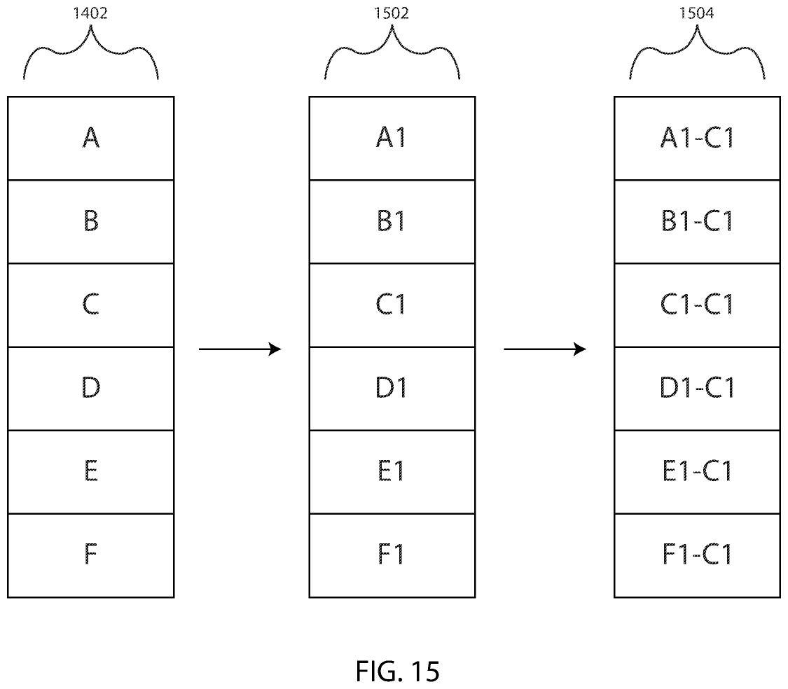

Referring now to FIG. 15, a schematic view of classification models is shown in accordance with various embodiments herein. In this embodiment, if the system can determine or be provided with certain characteristics of the individual wearing the ear-wearable device, instead of starting with a default machine learning classification model 1402, the ear-wearable system can start with a characteristic or demographic specific classification model 1502. Using that model as a starting point, the ear-wearable system can further modify/update the model based on data while the individual is wearing the device to generate a customized model 1504 for later use. In some embodiments, the customized model 1504 can be updated indefinitely.

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-wearable devices of the present disclosure can employ other radios, such as a 900 MHz radio or radios operating at other frequencies or frequency bands. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

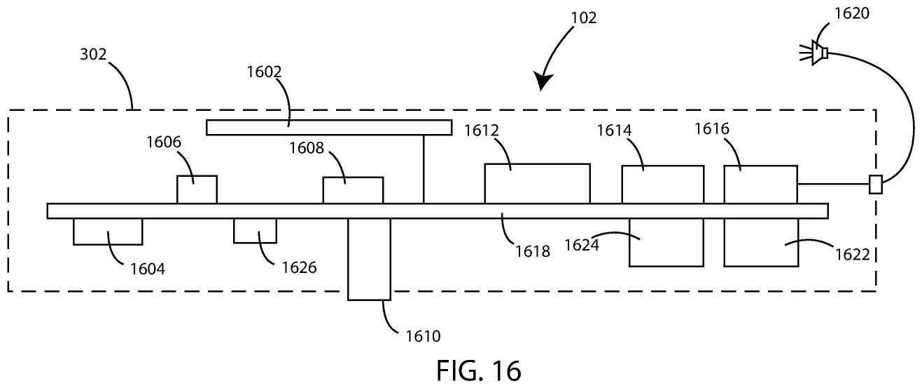

Referring now to FIG. 16, a schematic block diagram is shown with various components of an ear-wearable device in accordance with various embodiments. The block diagram of FIG. 16 represents a generic ear-wearable device for purposes of illustration. The ear-wearable device 102 shown in FIG. 16 includes several components electrically connected to a flexible mother circuit 1618 (e.g., flexible mother board) which is disposed within housing 302. A power supply circuit 1604 can include a battery and can be electrically connected to the flexible mother circuit 1618 and provides power to the various components of the ear-wearable device 102. One or more microphones 1606 are electrically connected to the flexible mother circuit 1618, which provides electrical communication between the microphones 1606 and a digital signal processor (DSP) 1612. Microphones herein can be of various types including, but not limited to, unidirectional, omnidirectional, MEMS based microphones, piezoelectric microphones, magnetic microphones, electret condenser microphones, and the like. Among other components, the DSP 1612 incorporates or is coupled to audio signal processing circuitry configured to implement various functions described herein. A sensor package 1614 can be coupled to the DSP 1612 via the flexible mother circuit 1618. The sensor package 1614 can include one or more different specific types of sensors such as those described in greater detail below. One or more user switches 1610 (e.g., on/off, volume, mic directional settings) are electrically coupled to the DSP 1612 via the flexible mother circuit 1618. It will be appreciated that the user switches 1610 can extend outside of the housing 302.

An audio output device 1616 is electrically connected to the DSP 1612 via the flexible mother circuit 1618. In some embodiments, the audio output device 1616 comprises a speaker (coupled to an amplifier). In other embodiments, the audio output device 1616 comprises an amplifier coupled to an external receiver 1620 adapted for positioning within an ear of a wearer. The external receiver 1620 can include an electroacoustic transducer, speaker, or loud speaker. The ear-wearable device 102 may incorporate a communication device 1608 coupled to the flexible mother circuit 1618 and to an antenna 1602 directly or indirectly via the flexible mother circuit 1618. The communication device 1608 can be a BLUETOOTH® transceiver, such as a BLE (BLUETOOTH® low energy) transceiver or other transceiver(s) (e.g., an IEEE 802.11 compliant device). The communication device 1608 can be configured to communicate with one or more external devices, such as those discussed previously, in accordance with various embodiments. In various embodiments, the communication device 1608 can be configured to communicate with an external visual display device such as a smart phone, a video display screen, a tablet, a computer, or the like.

In various embodiments, the ear-wearable device 102 can also include a control circuit 1622 and a memory storage device 1624. The control circuit 1622 can be in electrical communication with other components of the device. In some embodiments, a clock circuit 1626 can be in electrical communication with the control circuit. The control circuit 1622 can execute various operations, such as those described herein. In various embodiments, the control circuit 1622 can execute operations resulting in the provision of a user input interface by which the ear-wearable device 102 can receive inputs (including audible inputs, touch based inputs, and the like) from the device wearer. The control circuit 1622 can include various components including, but not limited to, a microprocessor, a microcontroller, an FPGA (field-programmable gate array) processing device, an ASIC (application specific integrated circuit), or the like. The memory storage device 1624 can include both volatile and non-volatile memory. The memory storage device 1624 can include ROM, RAM, flash memory, EEPROM, SSD devices, NAND chips, and the like. The memory storage device 1624 can be used to store data from sensors as described herein and/or processed data generated using data from sensors as described herein.

It will be appreciated that various of the components described in FIG. 16 can be associated with separate devices and/or accessory devices to the ear-wearable device. By way of example, microphones can be associated with separate devices and/or accessory devices. Similarly, audio output devices can be associated with separate devices and/or accessory devices to the ear-wearable device. Further accessory devices as discussed herein can include various of the components as described with respect to an ear-wearable device. For example, an accessory device can include a control circuit, a microphone, a motion sensor, and a power supply, amongst other things.

Accessory devices herein can include various different components. In some embodiments, the accessory device can be a personal communications device, such as a smart phone. However, the accessory device can also be other things such as a secondary wearable device, a handheld computing device, a dedicated location determining device (such as a handheld GPS unit), or the like.

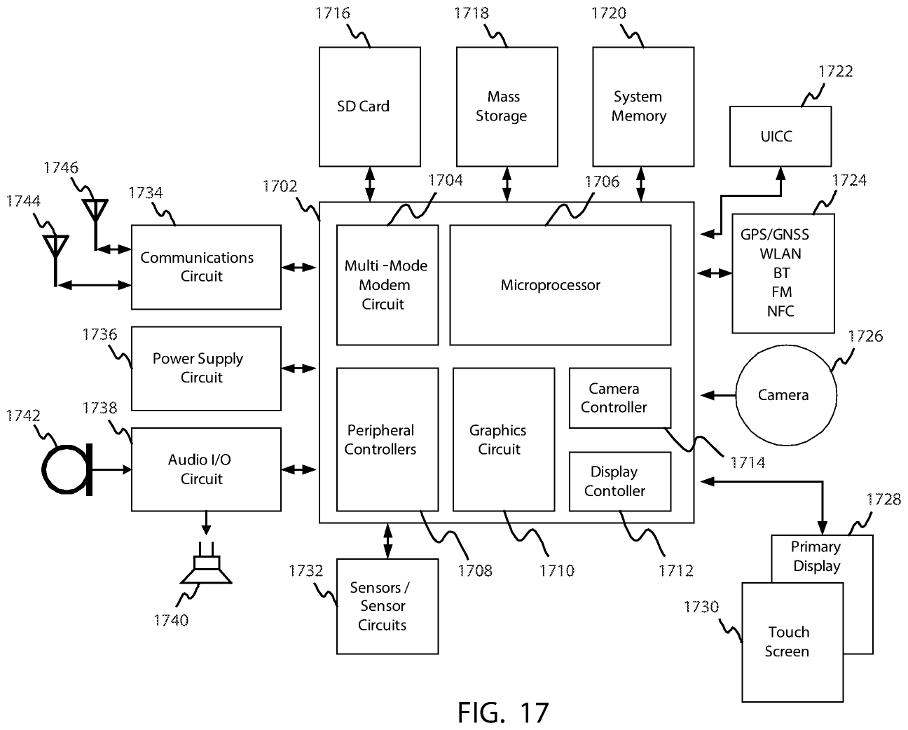

Referring now to FIG. 17, a schematic block diagram is shown of components of an accessory device (which could be a personal communications device or another type of accessory device) in accordance with various embodiments herein. This block diagram is just provided by way of illustration and it will be appreciated that accessory devices can include greater or lesser numbers of components. The accessory device in this example can include a control circuit 1702. The control circuit 1702 can include various components which may or may not be integrated. In various embodiments, the control circuit 1702 can include a microprocessor 1706, which could also be a microcontroller, FPGA, ASIC, or the like. The control circuit 1702 can also include a multi-mode modem circuit 1704 which can provide communications capability via various wired and wireless standards. The control circuit 1702 can include various peripheral controllers 1708. The control circuit 1702 can also include various sensors/sensor circuits 1732. The control circuit 1702 can also include a graphics circuit 1710, a camera controller 1714, and a display controller 1712. In various embodiments, the control circuit 1702 can interface with an SD card 1716, mass storage 1718, and system memory 1720. In various embodiments, the control circuit 1702 can interface with universal integrated circuit card (UICC) 1722. A spatial location determining circuit can be included and can take the form of an integrated circuit 1724 that can include components for receiving signals from GPS, GLONASS, BeiDou, Galileo, SBAS, WLAN, BT, FM, NFC type protocols, 5G picocells, or E911. In various embodiments, the accessory device can include a camera 1726. In various embodiments, the control circuit 1702 can interface with a primary display 1728 that can also include a touch screen 1730. In various embodiments, an audio I/O circuit 1738 can interface with the control circuit 1702 as well as a microphone 1742 and a speaker 1740. In various embodiments, a power supply or power supply circuit 1736 can interface with the control circuit 1702 and/or various other circuits herein in order to provide power to the system. In various embodiments, a communications circuit 1734 can be in communication with the control circuit 1702 as well as one or more antennas (1744, 1746).

It will be appreciated that in some cases a trend regarding PTSD symptoms may be more important than an instantaneous measure or snapshot of such symptoms. For example, an hour-long trend where detected PTSD symptoms rise to higher and higher levels may represent a greater health danger to an individual (and thus meriting intervention) than a brief spike in detected PTSD symptom levels. As such, in various embodiments herein the ear-wearable system 700 wherein the ear-wearable system 700 is configured to record data regarding occurrences of PTSD symptoms and calculate a trend regarding the same. The trend can span minutes, hours, days, weeks or months. Various actions can be taken by the system or device in response to the trend. For example, wherein the trend is upward (a trend toward increased PTSD symptoms) the device may initiate suggestions for corrective actions and/or increase the frequency with which such suggestions are provided to the device wearer. If suggestions are already being provided and/or actions are already being taken by the device and the trend is upward (a negative trend toward increased PTSD symptoms) the device may be configured to change the suggestions/instructions being provided to the device wearer as the current suggestions/instructions are being empirically shown to be ineffective.

In various embodiments, the ear-wearable system can be configured to generate and/or use a predicted PTSD symptom level of the device wearer in a subsequent time period. For example, in various embodiments, the ear-wearable system can be configured to cross-reference classified PTSD symptom levels against a calendar of the device wearer and predict PTSD symptom levels that may be reached during events upcoming on the calendar such as meetings. In some embodiments, the calendar information can be input into the system or device by the device wearer or another third party. In some embodiments, the calendar information can be supplied by an accessory device, such as a smart phone. In some embodiments, the calendar information can be retrieved using a calendar API. In some cases, the system can offer suggestions to the device wearer in order to prepare for such predicted episodes of PTSD symptoms.

In various embodiments, the ear-wearable system can be configured to execute an operation to remediate the trigger of the PTSD symptoms, independent of action of the device wearer. For example, many people find a quieter environment to be less triggering than a louder environment. In various embodiments, the ear-wearable system can execute an operation including reducing the volume of sounds exceeding a threshold value. In various embodiments, the ear-wearable system can reduce a volume sounds by actuating a vent or autovent. A vent or autovent feature of an ear-wearable device can be used to selectively seal off the ear canal creating acoustic separation from the ambient environment. Examples of vent features include, but are not limited to, those found in commonly owned U.S. patent application Ser. No. 13/720,793 (now issued as U.S. Pat. No. 8,923,543), entitled HEARING ASSISTANCE DEVICE VENT VALVE, and commonly-owned U.S. Provisional Patent Application No. 62/850,805, entitled SOLENOID ACTUATOR IN A HEARING DEVICE, both of which are hereby incorporated by reference herein in their entirety.

As another example of remediation or an action to mitigate PTSD symptoms, in various embodiments the ear-wearable system can execute an operation to reduce volume by adjusting a gain value of the ear-wearable system. As part of functionality associated with hearing assistance, devices herein can include amplifiers (digital or analog), filters (digital or analog), signal processing devices and the like. Operations of such devices can be altered to reduce a gain value so that sound provided to the device wearer via the ear-wearable device is reduced in volume. In this way, a sound that would otherwise be at a very high volume can be provided to the device wearer at a lesser volume, to reduce the impacts of the sound on the device wearer. In various embodiments, the ear-wearable system can execute an operation to change noise processing or noise reduction features associated with the ear-wearable device or system. For example, some transient noise (such as sounds that might be particularly annoying or alarming) can be reduced or otherwise suppressed by modifying the noise reduction features of the system or device. This can include, for example, ambient noise reduction/cancellation, frequency specific noise reduction/cancellation, or the like. In some embodiments, other aspects of device operation and/or therapy provision can be changed by the system as part of an effort to remediate and/or mitigate PTSD symptoms. For example, tinnitus can be treated with an auditory stimulus and in embodiments herein the nature of the stimulus or parameters of the stimulus can be changed in order to mitigate PTSD symptoms. As a specific example, in some cases, the tinnitus stimulus can switch to music instead of noise (or vice versa if the device wearer prefers it) in order to help the device wearer with their PTSD symptoms. Aspects of tinnitus therapy can be found in U.S. patent Ser. No. 10/537,268, the content of which is herein incorporated by reference.

### Pattern Identification

It will be appreciated that in various embodiments herein, a device or a system can be used to detect a pattern or patterns indicative of a trigger of PTSD symptoms or a worsening thereof. Also in various embodiments herein, a device or a system can be used to detect a pattern or patterns indicative of an occurrence of PTSD symptoms and/or symptoms of a specific level of intensity. Such patterns can be detected in various ways. Some techniques are described elsewhere herein, but some further examples will now be described.

As merely one example, one or more sensors can be operatively connected to a controller (such as the control circuit described in FIG. 17) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, machine learning approaches such as neural network models and deep learning, and the like.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as patterns indicative of indicative of an occurrence of a trigger or an occurrence of PTSD symptoms (positive example patterns), one or more predetermined patterns that service as patterns indicative of the absence of a trigger or an absence of PTSD symptoms (negative example patterns), or the like. As merely one scenario, if a pattern is detected in an individual that exhibits similarity crossing a threshold value to a particular positive example pattern or substantial similarity to that pattern, wherein the pattern is specific for a trigger, a PTSD symptom episode and/or a PTSD symptom episode of a specific level of intensity, then that can be taken as an indication of an occurrence of that type of event experienced by the device wearer.

Similarity and dissimilarity can be measured directly via standard statistical metrics such normalized Z-score, or similar multidimensional distance measures (e.g., Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the health status of an individual (and, in particular, their status with respect to triggers and/or PTSD symptoms), over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

Various embodiments herein specifically include the application of a machine learning classification model. In various embodiments, the ear-wearable system can be configured to periodically update the machine learning classification model based on indicators of triggers and/or PTSD symptoms experienced by the device wearer and/or by observing PTSD symptoms experienced by the device wearer as caused by particular potential triggers.

In some embodiments, a training set of data can be used in order to generate a machine learning classification model. The input data can include microphone and/or sensor data as described herein as tagged/labeled with binary and/or non-binary classifications of triggers and/or PTSD symptoms. Binary classification approaches can utilize techniques including, but not limited to, logistic regression, k-nearest neighbors, decision trees, support vector machine approaches, naive B ayes techniques, and the like. Multi-class classification approaches (e.g., for non-binary classifications of triggers and/or PTSD symptoms) can include k-nearest neighbors, decision trees, naive Bayes approaches, random forest approaches, and gradient boosting approaches amongst others.

In various embodiments, the ear-wearable system is configured to execute operations to generate or update the machine learning model on the ear-wearable device itself. In some embodiments, the ear-wearable system may convey data to another device such as an accessory device or a cloud computing resource in order to execute operations to generate or update a machine learning model herein. In various embodiments, the ear-wearable system is configured to weight certain possible detected indicators of triggers and/or PTSD symptoms in the machine learning classification model more heavily based on derived correlations specific for the individual as described elsewhere herein.

### Sensor Package

Various embodiments herein include a sensor package. Specifically, systems and ear-wearable devices herein can include one or more sensor packages (including one or more discrete or integrated sensors) to provide data for use with operations to characterize the PTSD symptoms experienced by an individual as well as characterize possible triggers. Further details about the sensor package are provided as follows. However, it will be appreciated that this is merely provided by way of example and that further variations are contemplated herein. Also, it will be appreciated that a single sensor may provide more than one type of physiological data. For example, heart rate, respiration, blood pressure, or any combination thereof may be extracted from PPG sensor data.

In various embodiments, the indicators of PTSD symptoms experienced by the device wearer are derived from data produced by at least one of the microphone and the sensor package. In various embodiments, the sensor package can include at least one including at least one of a heart rate sensor, a heart rate variability sensor, an electrocardiogram (ECG) sensor, a blood oxygen sensor, a blood pressure sensor, a skin conductance sensor, a photoplethysmography (PPG) sensor, a temperature sensor (such as a core body temperature sensor, skin temperature sensor, ear-canal temperature sensor, or another temperature sensor), a motion sensor, an electroencephalograph (EEG) sensor, and a respiratory sensor. In various embodiments, the motion sensor can include at least one of an accelerometer and a gyroscope.

The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motions sensors can be used to track movements of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer (or barometric pressure sensor), an altimeter, a magnetometer, a magnetic sensor, an eye movement sensor, a pressure sensor, an acoustic sensor, a telecoil, a heart rate sensor, a global positioning system (GPS), a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, a sympathetic nervous stimulation sensor (which in some embodiments can including other sensors described herein to detect one or more of increased mental activity, increased heart rate and blood pressure, an increase in body temperature, increased breathing rate, or the like), eye movement sensor (e.g., electrooculogram (EOG) sensor), myographic potential electrode sensor (or electromyography—EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor, and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, a visible light sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of another medical device, e.g., an insulin pump device or a heart pacemaker device.

### Own Voice Detection

Distinguishing between speech or sounds associated with the device wearer and speech or sounds associated with a third party can be performed in various ways. In some embodiments, this can be performed through signal analysis of the signals generated from the microphone(s). For example, in some embodiments, this can be done by filtering out frequencies of sound that are not associated with speech of the device-wearer. In some embodiments, such as where there are two or more microphones (on the same ear-wearable device or on different ear-wearable devices) this can be done through spatial localization of the origin of the speech or other sounds and filtering out, spectrally subtracting, or otherwise discarding sounds that do not have an origin within the device wearer. In some embodiments, such as where there are two or more ear-worn devices, own-voice detection can be performed and/or enhanced through correlation or matching of intensity levels and or timing.

In some cases, the system can include a bone conduction microphone to preferentially pick up the voice of the device wearer. In some cases, the system can include a directional microphone that is configured to preferentially pick up the voice of the device wearer. In some cases, the system can include an intracanal microphone (a microphone configured to be disposed within the ear-canal of the device wearer) to preferentially pick up the voice of the device wearer. In some cases, the system can include a motion sensor (e.g., an accelerometer configured to be on or about the head of the wearer) to preferentially pick up skull vibrations associated with the vocal productions of the device wearer.

In some cases, an adaptive filtering approach can be used. By way of example, a desired signal for an adaptive filter can be taken from a first microphone and the input signal to the adaptive filter is taken from the second microphone. If the hearing aid wearer is talking, the adaptive filter models the relative transfer function between the microphones. Own-voice detection can be performed by comparing the power of an error signal produced by the adaptive filter to the power of the signal from the standard microphone and/or looking at the peak strength in the impulse response of the filter. The amplitude of the impulse response should be in a certain range to be valid for the own voice. If the user's own voice is present, the power of the error signal will be much less than the power of the signal from the standard microphone, and the impulse response has a strong peak with an amplitude above a threshold. In the presence of the user's own voice, the largest coefficient of the adaptive filter is expected to be within a particular range. Sound from other noise sources results in a smaller difference between the power of the error signal and the power of the signal from the standard microphone, and a small impulse response of the filter with no distinctive peak. Further aspects of this approach are described in U.S. Pat. No. 9,219,964, the content of which is herein incorporated by reference.

In another approach, the system uses a set of signals from a number of microphones. For example, a first microphone can produce a first output signal A from a filter and a second microphone can produce a second output signal B from a filter. The apparatus includes a first directional filter adapted to receive the first output signal A and produce a first directional output signal. A digital signal processor is adapted to receive signals representative of the sounds from the user's mouth from at least one or more of the first and second microphones and to detect at least an average fundamental frequency of voice (pitch output) F₀. A voice detection circuit is adapted to receive the second output signal B and the pitch output F₀ and to produce an own voice detection trigger T. The apparatus further includes a mismatch filter adapted to receive and process the second output signal B, the own voice detection trigger T, and an error signal E, where the error signal E is a difference between the first output signal A and an output O of the mismatch filter. A second directional filter is adapted to receive the matched output O and produce a second directional output signal. A first summing circuit is adapted to receive the first directional output signal and the second directional output signal and to provide a summed directional output signal (D). In use, at least the first microphone and the second microphone are in relatively constant spatial position with respect to the user's mouth, according to various embodiments. Further aspects of this approach are described in U.S. Pat. No. 9,210,518, the content of which is herein incorporated by reference.

In various embodiments herein, a device or system can specifically include an inward-facing microphone (e.g., facing the ear canal, or facing tissue, as opposed to facing the ambient environment.) A sound signal captured by the inward-facing microphone can be used to determine physiological information, such as that relating to a physiological response indicative of PTSD symptoms. For example, a signal from an inward-facing microphone may be used to determine heart rate, respiration, or both, e.g., from sounds transferred through the body. In some examples, a measure of blood pressure may be determined, e.g., based on an amplitude of a detected physiologic sound (e.g., louder sound correlates with higher blood pressure.)

### Methods

Many different methods are contemplated herein, including, but not limited to, methods of making devices, methods of using devices, methods of detecting PTSD triggers or PTSD symptoms, methods of monitoring PTSD symptoms, methods of treating PTSD or PTSD symptoms, or preventing PTSD episodes, and the like. Aspects of system/device operation described elsewhere herein can be performed as operations of one or more methods in accordance with various embodiments herein.

In some embodiments, a method of monitoring an individual using an ear-wearable stress monitoring system is included, the method including evaluating data from at least one of a microphone and a sensor package, classifying a PTSD symptom level of the individual using a machine learning classification model, and updating the machine learning classification model based on indicators of PTSD symptoms experienced by the individual.

In some embodiments, a method can further include deriving triggers of PTSD symptoms experienced by the device wearer from data produced by at least one of the microphone and the sensor package. In an embodiment, the method can further include deriving triggers of PTSD symptoms experienced by the device wearer from input provided by the individual. In an embodiment, the method can further include deriving triggers of PTSD symptoms experienced by the device wearer from input provided by an external device.

In some embodiments, a method can further include correlating possible detected triggers with subsequent PTSD symptoms of the device wearer. In an embodiment of the method, correlating possible detected triggers with subsequent PTSD symptoms of the device wearer further comprises weighting certain possible detected triggers in the machine learning classification model more heavily based on the correlation.

In some embodiments, a method can further include sending information regarding triggers and/or PTSD symptoms and/or episodes to an electronic medical record system. In some embodiments, the method can further include sending information regarding the same to a third party.

In some embodiments, a method can further include providing instructions to an individual regarding an action to take to address or ameliorate PTSD symptoms of the individual. In an embodiment of the method, the instructions to the individual comprise breathing instructions. In some embodiments of the method, the instructions to the individual comprise a suggestion to take a medication.

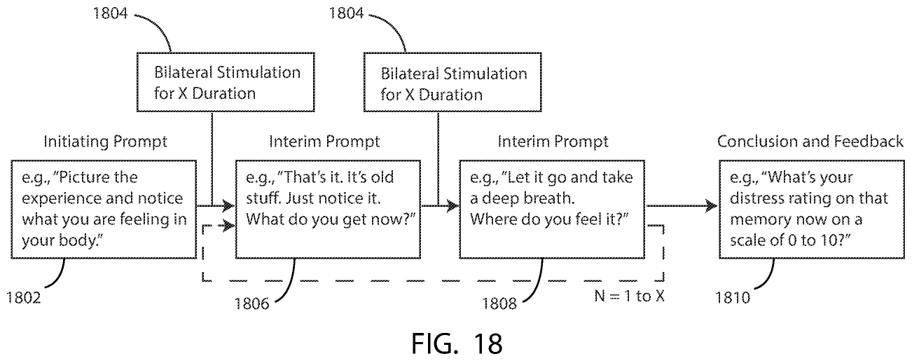

In some embodiments, a method can include initiating administration of desensitization and reprocessing (DR) therapy to a device wearer. Referring now to FIG. 18, a flow chart of operations associated with administration of a therapy herein is shown as an example. The process of administering DR therapy can involve alternating prompts 1802, 1806, 1808 for the user and then providing bilateral stimulation 1804 while processing the target memory. The period of bilateral stimulation typically lasts from 30 seconds to several minutes, but can last for shorter or longer periods. The prompts can be general defaults as depicted in FIG. 18, or they may be customized by the device wearer or other individual (e.g., caregiver, psychologist, psychiatrist, etc.). The prompts can be presented to the user acoustically via device receiver, visually displayed on accessory device, or both. The application of bilateral and/or binaural stimulation and interim prompts can be repeated a number of times. The overall therapy session can last for 2, 5, 10, 15, 20, 30, 45, 60, 75, 90, or 120 minutes of more, or an amount of time falling within a range between any of the foregoing.

To conclude the therapy session, a final prompt soliciting user feedback 1810, such as rating the emotional valence of the target memory can be provided to the device wearer. The device wearer can submit their feedback acoustically (i.e., the device wearer can say answer aloud, and that response can be picked up by device microphone), gesturally (i.e., head nods, or sign language interpreted by the camera), or directly logged to a device, such as an accessory device, such as by touchscreen input, manual button presses on the hearing device, or finger taps sensed by the motion sensor.

In some embodiments, a method can include monitoring engagement by directly sensing device wearer activity. In some embodiments, a method can include monitoring engagement by indirectly monitoring sustained attention. In some embodiments, a method can include automatically adjusting properties of binaural stimulation to maintain device wearer engagement.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable stress therapy system comprising:
a control circuit;
a first sensor package, wherein the first sensor package is in electrical communication with the control circuit;
a microphone, wherein the microphone is in electrical communication with the control circuit; and
an electroacoustic transducer, wherein the electroacoustic transducer is in electrical communication with the control circuit;
wherein the ear-wearable stress therapy system is configured to initiate administration of desensitization and reprocessing (DR) therapy to a device wearer.

2. The ear-wearable stress therapy system of claim 1, wherein the ear-wearable stress therapy system is configured to administer and control delivery of binaural stimulation as part of the DR therapy.

3-6. (canceled)

7. The ear-wearable stress therapy system of claim 2, wherein the ear-wearable stress therapy system is configured to automatically adjust properties of the binaural stimulation to maintain device wearer engagement.

8-10. (canceled)

11. The ear-wearable stress therapy system of claim 1, wherein the ear-wearable stress therapy system is configured to detect post-traumatic stress disorder (PTSD) symptoms.

12. The ear-wearable stress therapy system of claim 11, wherein the ear-wearable stress therapy system is configured to initiate administration of desensitization and reprocessing (DR) therapy to a device wearer subsequent to detection of PTSD symptoms.

13. The ear-wearable stress therapy system of claim 11, wherein the ear-wearable stress therapy system is configured to record microphone and/or sensor data over a look-back period when PTSD symptoms are detected.

14. The ear-wearable stress therapy system of claim 13, wherein the ear-wearable stress therapy system is configured to use the recorded microphone and/or sensor data to characterize at least one of a sound environment of the device wearer, a physical activity level of the device wearer, a conversation pattern of the device wearer, a level of irritability of the device wearer, an aggression level of the device wearer, and an emotional state of the device wearer.

15. The ear-wearable stress therapy system of claim 14, wherein the ear-wearable stress therapy system is configured to identify a triggering event selected from at least one a sound environment, a physical geolocation, a physical activity, and a conversation pattern.

16-18. (canceled)

19. The ear-wearable stress therapy system of claim 11, wherein the detection of the PTSD symptoms is based on at least one of a heart rate, a blood pressure, a physiologic temperature, the device wearer's voice, and a motion pattern.

20. The ear-wearable stress therapy system of claim 11, wherein the ear-wearable stress therapy system is configured to detect medication administration events and determine a correlation between detected medication administration events and detected PTSD symptoms.

21. The ear-wearable stress therapy system of claim 20, wherein the ear-wearable stress therapy system is configured to titrate medication dosage based on an observed correlation between detected medication administration events and detected PTSD symptoms.

22. The ear-wearable stress therapy system of claim 11, wherein the ear-wearable stress therapy system is configured to provide audio stimulation when PTSD symptoms are detected.

23-24. (canceled)

25. The ear-wearable stress therapy system of claim 11, wherein the ear-wearable stress therapy system is configured to query the device wearer about their perceived stress level when PTSD symptoms are detected.

26. (canceled)

27. The ear-wearable stress therapy system of claim 1, wherein the ear-wearable stress therapy system is configured to detect a PTSD symptom causing trigger.

28. The ear-wearable stress therapy system of claim 27, wherein the ear-wearable stress therapy system is configured to initiate administration of desensitization and reprocessing (DR) therapy to a device wearer when a PTSD symptom causing trigger has been detected.

29. (canceled)

30. The ear-wearable stress therapy system of claim 27, wherein the ear-wearable stress therapy system is configured to attenuate the PTSD symptom causing trigger by modulating an output from the electroacoustic transducer.

31. The ear-wearable stress therapy system of claim 30, wherein the PTSD symptom causing trigger is attenuated by reducing a volume of sound outputted by the electroacoustic transducer.

32-35. (canceled)

36. The ear-wearable stress therapy system of claim 1, wherein the ear-wearable stress therapy system is configured to detect a startle response.

37. The ear-wearable stress therapy system of claim 36, wherein the ear-wearable stress therapy system is configured to detect a startle response based on at least one of motion sensor signals, microphone signals, and photoplethysmography sensor signals.

38-43. (canceled)

44. The ear-wearable stress therapy system of claim 1, wherein the ear-wearable stress therapy system is configured to detect a level of social engagement of the device wearer.

45-50. (canceled)
